(19) United States
(12) Patent Application Publication
(10) Pub. No.: US 2025/0116665 A1
(43) Pub. Date: Apr. 10, 2025

(54) ANALYSIS SYSTEM AND METHOD OF ANALYSIS

(71) Applicant: Georgia Tech Research Corporation, Atlanta, GA (US)

(72) Inventors: Austin Lance Culberson, Atlanta, GA (US); Mason Chilmonczyk, Atlanta, GA (US); Andrei G. Fedorov, Atlanta, GA (US); Peter Arthur Kottke, Atlanta, GA (US)

(21) Appl. No.: 18/695,545

(22) PCT Filed: Sep. 29, 2022
(86) PCT No.: PCT/US2022/077220

§ 371 Date: Mar. 26, 2024

Related U.S. Application Data
(60) Provisional application No. 63/250,367, filed on Sep. 30, 2021.

(51) Int. Cl. G01N 33/569; G01N 33/68; H01J 49/16

(52) U.S. Cl. CPC G01N 33/56972; G01N 33/6851; H01J 49/165

(57) ABSTRACT

The present disclosure provides for analysis systems and methods of analyzing a sample. The present disclosure provides for acquiring a sample that includes targeted cargo-containing object (e.g., cell), trapping the targeted cargo-containing object, releasing the cargo of the targeted cargo containing object, and analyzing the cargo free of the signal deteriorating media. The analysis system can include a sample introduction system, an object cargo extraction system and a detection system. The sample introduction system acquires the sample (e.g., directly or indirectly).

### CLAIM OF PRIORITY TO RELATED APPLICATION

This application claims priority to co-pending U.S. provisional application entitled “MICROFLUIDIC PLATFORM FOR INTRACELLULAR ANALYSIS” having Ser. No. 63/250,367 filed on Sep. 30, 2021, which is entirely incorporated herein by reference.

### STATEMENT REGARDING FEDERALLY SPONSORED RESEARCH OR DEVELOPMENT

This invention was made with government support under grant 1648035 awarded by the National Science Foundation. The government has certain rights in the invention.

### BACKGROUND

Analysis of the intracellular environment provides unparalleled insight into the cellular state including differentiation stage, metabolic state, and overall health. Conventional intracellular workflows require tedious, manual, wasteful, and time-consuming sample preparation. Thus, there is a need for better technology.

### SUMMARY

The present disclosure provides for analysis systems and methods of analyzing a sample. The present disclosure provides for acquiring a sample that includes a targeted cargo-containing object(s) (e.g., cell), trapping the targeted cargo-containing object, releasing the cargo of the targeted cargo-containing object, and analyzing the cargo free of the signal deteriorating media. The analysis system can include a sample introduction system, an object cargo extraction system and a detection system. The sample introduction system acquires the sample (e.g., directly or indirectly). The sample includes the targeted cargo-containing objects (e.g., cells) and other materials that can be present in the sample (e.g., extra-media material (e.g., extracellular material)). The object cargo extraction system traps the targeted cargo-containing objects (and removes other materials) and separates the cargo from the targeted cargo-containing objects, where the cargo can be flowed to the detection system. The detection system can detect the presence and/or concentration of the cargo and/or other components.

In an aspect, the present disclosure provides for an analysis system comprising: a sample introduction system configured to deliver a sample, wherein the sample introduction system includes an inlet and an outlet, wherein the sample includes one or more targeted cargo-containing objects; an object cargo extraction system, wherein the object cargo extraction system includes an inlet and an outlet, wherein a channel includes one or more capture features positioned between the inlet and the outlet, wherein the one or more capture features are configured to trap the targeted cargo-containing objects in the sample as the sample flows through the channel from the inlet to the outlet, wherein electrodes are positioned adjacent to an area of the channel, wherein the electrodes are configured for electrical lysis of targeted cargo-containing objects present in the area of the channel, wherein the inlet of the object cargo extraction system is in fluidic communication with the outlet of the sample introduction system; and a detection system, wherein the object cargo extraction system is in fluidic communication with the detection system, wherein the detection system is configured to detect cargo released from the targeted cargo-containing objects.

In an aspect, the present disclosure provides for a method of analysis, comprising: introducing a sample to an object cargo extraction system, wherein the sample includes extra-object media and targeted cargo-containing objects, wherein the object cargo extraction system includes a channel, an inlet, an outlet, and a one or more capture features positioned between the inlet and the outlet, wherein the one or more capture features trap the targeted cargo-containing objects in an area of the channel, wherein the object cargo extraction system includes electrodes positioned adjacent the channel, wherein the electrodes are configured for electrical lysis of targeted cargo-containing objects present in the area of the channel; applying one or more electrical pulse across the electrodes to lyse the targeted cargo-containing objects, wherein the cargo present in the targeted cargo-containing objects pass through the one or more capture features, optionally wherein other debris from the lysing of the targeted cargo-containing object do not pass through the one or more capture features; and flowing the cargo out of the outlet to a detection system, wherein the detection system is configured to identify the one or more of the cargo.

### BRIEF DESCRIPTION OF THE DRAWINGS

Many aspects of the present disclosure can be better understood with reference to the following drawings. The components in the drawings are not necessarily to scale, with emphasis instead being placed upon clearly illustrating the principles of the disclosure. Moreover, in the drawings, like reference numerals designate corresponding parts throughout the several views.

FIG. 1.1 illustrates a schematic of an embodiment of the analysis system.

FIG. 1.2 illustrates a schematic of a more detailed embodiment of the analysis system.

FIG. 1.3 illustrates a schematic of an embodiment of the in-line introduction of the sample.

FIG. 1.4 illustrates a schematic of an embodiment of the at-line introduction of the sample.

FIG. 1.5 illustrates a schematic of a portion of the object cargo extraction system.

FIGS. 1.6A-6E illustrate schematics of configurations of the object capture features.

FIG. 2.1 illustrates conventional mass spectrometry intracellular metabolomics workflows (top) which require time consuming sample preparation of large numbers of cells in manually prepared batches. An embodiment of the present disclosure (bottom) enables direct from culture sampling, automated sample preparation in a microfluidic device, and rapid ESI-MS. The sample-to-analysis integration removes manual handling, reduces analysis time, and enables analysis of small numbers of cells.

FIG. 2.2 illustrates a schematic of the present disclosure that corresponds to the aspects of FIGS. 1.1 and 1.2. FIG. 2.2 illustrates the integrated sample-to-analysis platform is composed of a sampling interface (left) (e.g., sample introduction system 200), cell processing device (middle) (e.g., object cargo extraction system 300), and ESI-MS interface (right) (detection system 400). The analysis process begins with uptake of a cell laden sample directly from the cell system (e.g., petri dish, bioreactor, or vial). The sample plug is loaded into the microfabricated cell processing device (b). The silicon-based device is comprised of a microfluidic channel, cell immobilization features, and integrated electrodes. The device is sealed by a transparent, Borofloat cover bonded using an SU-8 adhesive layer. This allows the channel to be inspected via digital microscope throughout cell processing. In the device, cells are immobilized, rinsed, and lysed in the processing zone (c). During rinsing, the extracellular matrix is directed to waste; upon lysis, the downstream flow is diverted to the ESI interface for direct infusion ESI-MS. The system is then reconditioned by back flowing rinsing buffer via a secondary purge pump to remove cell debris from the microfluidic device prior to subsequent analyses.

FIG. 2.3 illustrates ESI-MS intracellular amino acid detection depicted as protonated monoisotopic m/z traces during the period immediately following lysis. The amino acids are sorted from most hydrophobic at top to most hydrophilic at bottom (at neutral pH). Recording of the MS signal begins following the rinsing step, approximately 4 minutes post sample uptake. The traces are shown immediately following lysis corresponding to 2 minutes post rinse or 6 minutes post sample uptake. The traces are normalized for each analyte with the maximum signal intensity given in parenthesis for the displayed time range.

FIG. 2.4 illustrates ESI-MS intracellular metabolite detection of relevance to HUVECs depicted as m/z traces during the period immediately following lysis. The metabolites are sorted by descending monoisotopic mass (left). The traces are normalized for each analyte by the maximum signal intensity (given in parenthesis) for the displayed time range. * Denotes fragments reported in MassBank of North America (MoNA), ** denotes fragments reported in MassBank Europe, ***denotes fragments reported in MZMine.

FIGS. 2.5A-2.5C illustrates the cell processing device (FIG. 2.5A) is comprised of a microfluidic channel (a1) in a silicon base with integrated electrodes (a2) along the channel. The channel is sealed with a transparent Borofloat cover while allowing the ends of the electrodes to remain exposed to facilitate connecting to the lysis circuit. A series of 5 μm wide pillars spans the microfluidic channel to prevent cells from passing further downstream and allow for rinsing prior to intracellular cargo extraction (FIG. 2.5B). FIG. 2.5C (top) illustrates a detailed view of the backside counterbore which serves as the zero-dead volume connection between the device and inlet/outlet tubing. FIG. 2.5C (bottom) illustrates a detailed view of the inlet and cell channel.

FIG. 2.6 illustrates the electrical lysis circuit design. Leads are connected to each electrode lining the channel to provide potential difference sufficient for lysis according to the chosen pulse parameters. A high voltage IGBT enables rapid switching to open or close the circuit, allowing for one electrode to effectively drain or float based on the voltage drop across the resistance network. The switch allows for the system to be electrically isolated from the ESI circuit until lysis pulses are applied.

FIG. 2.7 illustrates cell processing device fabrication sequence. Devices are batch fabricated with 32 devices on a 4″ wafer; the sequence shown is for a cross-section of a single device. As the electrodes are deposited along the channel sides, they are not shown past step A10; see FIG. 2.2 and FIGS. 2.5A-C for alternative views of the device.

FIG. 2.8 illustrates Taylor-Aris dispersion modelling informs optimal flowrate, tube diameter, and tube lengths to maintain small concentrations of analytes resulting from the small lysate volume while balancing time and pressure drop considerations. (Top) Plot of effective diffusion coefficient vs flowrate for a 10 cm length of tubing shows that dispersion becomes dominate means of diffusive transport at higher flowrates. (Middle) Plot of mean concentration (normalized Cm/Co where Co is initial concentration) vs flowrate for 10 cm length of tubing shows that dilution curves collapse for microfluidic tube diameters (10-100 μm ID) at nano-ESI flowrates (1-10 nL/s). (Bottom) Plot of mean concentration vs flowrate for varying tube lengths for a 100 μm ID tube highlights importance of minimizing tube length to mitigate dilution effects. Reducing length also reduces transit time and pressure drop. For reference the experimental flowrate was 30 μL/hr (8.3 nL/s).

FIG. 2.9 illustrates both methionine and arginine show little variation in the protonated monoisotopic mass traces (top trace) but display distinct signal increases in multiple fragments reported in the MassBank Europe database for ESI-MS. The traces are normalized by the maximum signal intensity for the displayed time range; the maximum signal intensity and corresponding mass are provided to the right of each trace. These results were obtained from an embodiment of the present disclosure.

FIG. 2.10 illustrates the NH4+ adduct of phenylalanine (right) displayed a distinct signal increase compared to the protonated monoisotopic mass trace (left). The traces are normalized by the maximum signal intensity for the displayed time range. These results were obtained from an embodiment of the present disclosure.

FIG. 2.11 illustrates representative spectra of both detected and undetected metabolites. For each analyte, the target m/z is shown for time periods before (top), during (middle), and after (bottom) lysate elution; the spectra are averaged over 15 seconds for each time point. Signal to noise (SN) values are provided for each m/z marker with the detected signals circled in red; tryptophan was not detected. The target m/z value is listed below each sub-figure and denoted as either a protonated monoisotopic mass or fragment mass; instrument error was taken as 10 ppm for the analysis. These results were obtained from an embodiment of the present disclosure.

FIG. 2.12 illustrates traces of the protonated monoisotopic mass of each amino acid to highlight the dependence of detection limits on number of cells loaded. The traces are normalized for each analyte by the maximum signal intensity (given in parenthesis) for the displayed time range. These results were obtained from an embodiment of the present disclosure.

FIGS. 3.1A-3.1C illustrate different embodiments of the present disclosure. These embodiments correspond to aspects shown in FIGS. 1.1, 1.2, and 2.2.

FIG. 3.2 illustrates SEM images of as fabricated immobilization feature designs including (FIG. 3.2A) single step and (FIG. 3.2B) pillars in parallel designs. Flow is right to left.

FIGS. 3.3A-D illustrate 2D models of cell immobilization feature designs leveraging the change in cortical tension as cells enter restrictions. FIG. 3.3A illustrates restriction features in parallel with alternating location of always open channel between subsequent rows. FIG. 3.3B illustrates flow velocity and streamlines of parallel configuration. FIG. 3.3C illustrates restriction features angled with alternating location of always open channel between subsequent rows. FIG. 3.3D illustrates flow velocity and streamlines of angled configuration. All flow values are relative as 2D model does not fully capture velocity magnitude of 3D configuration. Pillars span channel depth. Modelling and simulation performed in COMSOL Multiphysics 5.6.

FIG. 3.4 illustrates operating regime for successful cell capture assuming a 10 μm diameter cell, 35 pN/μm nominal cortical tension, and 15 μL/hr flowrate. Regions representing successful operating regimes are shown assuming an 11 μm always open channel. Region 1 represents gap sizes in which the pressure threshold for successful cell capture will not be exceeded by the pressure drop across individual capture features. Region 2 represents gap sizes in which the pressure drop across the always open channel is greater than the pressure drop across individual features. Region 3 represents gap sizes in which the pressure drop across the always open channel is less than the pressure threshold for successful capture. The intersection of regions 1, 2, and 3 represent the composite operating regime in which cells will be successfully captured during initial load and remain captured once all capture sites are occupied.

FIG. 3.5 illustrates the comparison of rinsing buffers on cytosol stability immediately following rinse (top) and after 10 minutes (bottom).

FIG. 3.6 illustrates cell experiments show ability to detect a broad range of intracellular analytes in under 10 minutes using the simplified microfluidic workflow.

FIGS. 3.7A-C illustrate dimensionality reduction analyses of mass spectrometry data obtained via the sample-to-analysis system reveals clear differentiation in the dynamics of T-cell activation. FIG. 3.7A illustrates PCA of activated and unactivated spectra over the first 48 hours showing dynamics of cell recovery from thaw and activation. FIG. 3.7B illustrates PCA of the activated condition at early vs late time points shows distinct separation with no overlap between the 95% confidence intervals. FIG. 3.7C illustrates PLS-DA of the activated vs unactivated condition at late time points shows distinct separation with no overlap between the 95% confidence intervals. All input spectra are full scan spectra normalized by their sum.

### DETAILED DESCRIPTION

Before the present disclosure is described in greater detail, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit (unless the context clearly dictates otherwise), between the upper and lower limit of that range, and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of biochemistry, biology, flow dynamics, analytical chemistry, microfabrication, and the like, which are within the skill of the art. Such techniques are explained fully in the literature.

The following examples are put forth so as to provide those of ordinary skill in the art with a complete disclosure and description of how to perform the methods and use the compositions and compounds disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is in atmosphere. Standard temperature and pressure are defined as 25° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

### Discussion:

Embodiments of the present disclosure provide for analysis systems and methods of analyzing a sample. The present disclosure provides for acquiring a sample that includes targeted cargo-containing object (e.g., cell), trapping the targeted cargo-containing object, releasing the cargo of the targeted cargo-containing object, and analyzing the cargo free of the signal deteriorating media. In an aspect, the analysis system can include a sample introduction system, an object cargo extraction system and a detection system. The sample introduction system acquires the sample (e.g., directly or indirectly). The sample includes the targeted cargo-containing objects (e.g., cells) and other materials that can be present in the sample (e.g., extra-media material (e.g., extracellular material and secreted materials)). The object cargo extraction system traps the targeted cargo-containing objects (and can separate or remove other material) and separates the cargo from the targeted cargo-containing objects, where the cargo can be flowed to the detection system. The detection system can detect the presence and/or concentration of the cargo and/or other components, which can include molecules (e.g., biomolecules such as biomarkers (e.g., metabolites, proteins, peptides, cytokines, growth factors, DNA, RNA, lipids)) that are present in targeted cargo-containing object or associated with the sample such as cells, extracellular vesicles, organelles, bacteria, viruses, synthetic membrane-bound particles, or synthetic scaffolds to which cells are bound. The present disclosure can be used in applications such as biomanufacturing (e.g., biologics production, cell and gene therapy production).

Aspects of the present disclosure can include a microfluidic platform to replace the numerous manual, time consuming, and variable steps of conventional intracellular metabolomics workflows. The present disclosure includes a relatively simple microfluidic workflow for rapid, in situ analysis of targeted cargo-containing objects such as cell culture systems. The analysis system can reduce the sample-to-analysis time to analyze small numbers of targeted cargo-containing objects (e.g., one or more cells) while maintaining the ability to detect low concentration intracellular analytes. While some descriptions of the present disclosure refer to targeted cargo-containing objects as “cells”, this is done for efficiency and it is understood that other types of targeted cargo-containing objects can replace “cells”, so the disclosure is not limited to only cells.

In general, elements of the present disclosure can include: (1) a sampling interface being comprised of a non-fouling, non-invasive, spatially resolved inlet for localized sampling designed to interface directly with a multitude of cell culture systems (for example), (2) a microfluidic processing device (e.g., object cargo extraction system) with integrated cell immobilization, rinsing, and lysis capabilities, (3) optional in-line lysate conditioning systems for liquid-liquid, liquid-solid, or capillary electrophoretic separation, (4) detection system such as an electrospray ionization interface, for example, for direct infusion of conditioned lysate for mass spectrometry analysis, and (5) fluidic components including pumps and valves to provide sample uptake, rinsing, analysis, and reconditioning flows with automation capabilities.

The present disclosure can be advantageous for one or more of the following. The analysis system and method are non-invasive/non-destructive. The sample volumes are small enough to be considered negligible compared to cell culture systems and are removed in a sterile manner so as not to compromise the culture process allowing for frequent, in-process monitoring. The method is rapid. The microfluidic design with integrated detection interface reduces processing time compared to conventional intracellular workflows. The analysis system and method can be automated by use of digital valves that enables fully automated or pushbutton control, removing all manual handling and associated variability. The analysis system is drop-in in that it can seamlessly link cell culture system with analysis instrument for a multitude of culture/instrument combinations for in-line use while also providing at-line capabilities. The analysis system and method are sensitive, where rinsing and controlled lysis in minimal dead volume device mitigates dilution of low abundance intracellular analytes while also minimizing effects of interfering compounds. The analysis system is reusable, where the components and devices can be reconditioned after each sample run, allowing for continuous use following a single installation.

Now having described components of the present disclosure generally, additional details are provided below and in Examples 1 and 2.

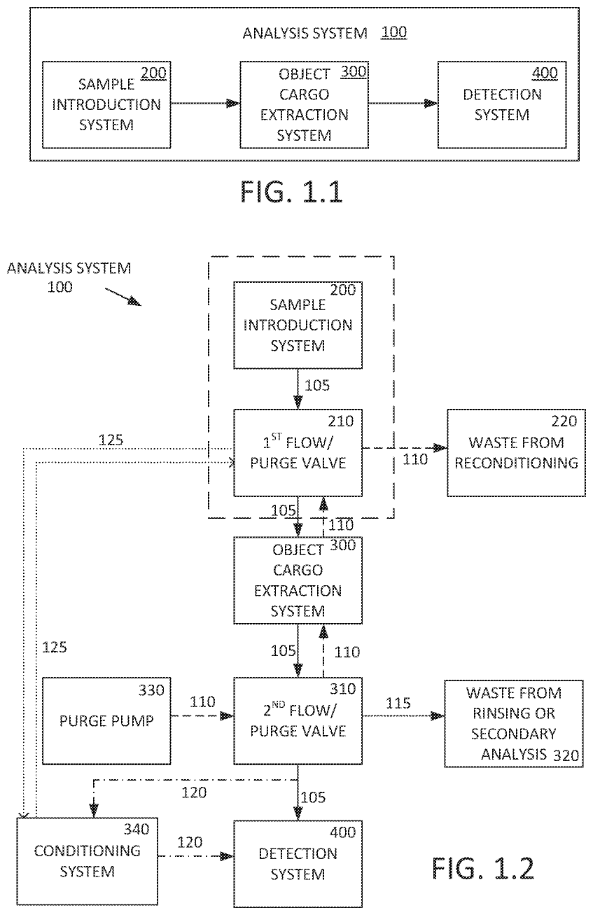

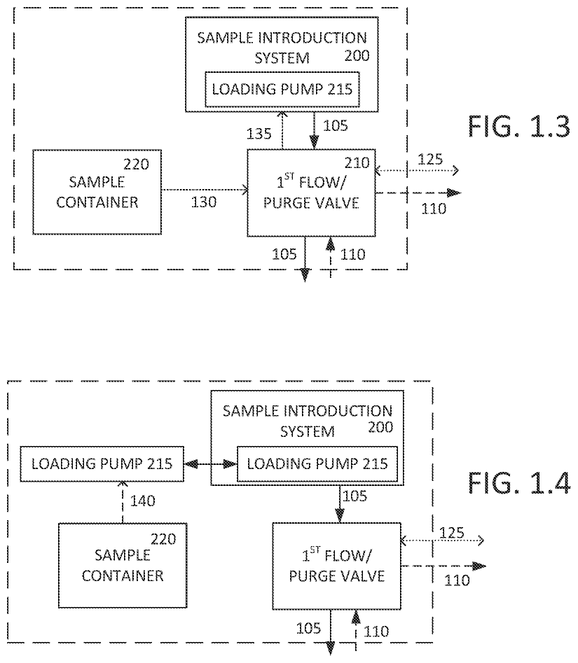

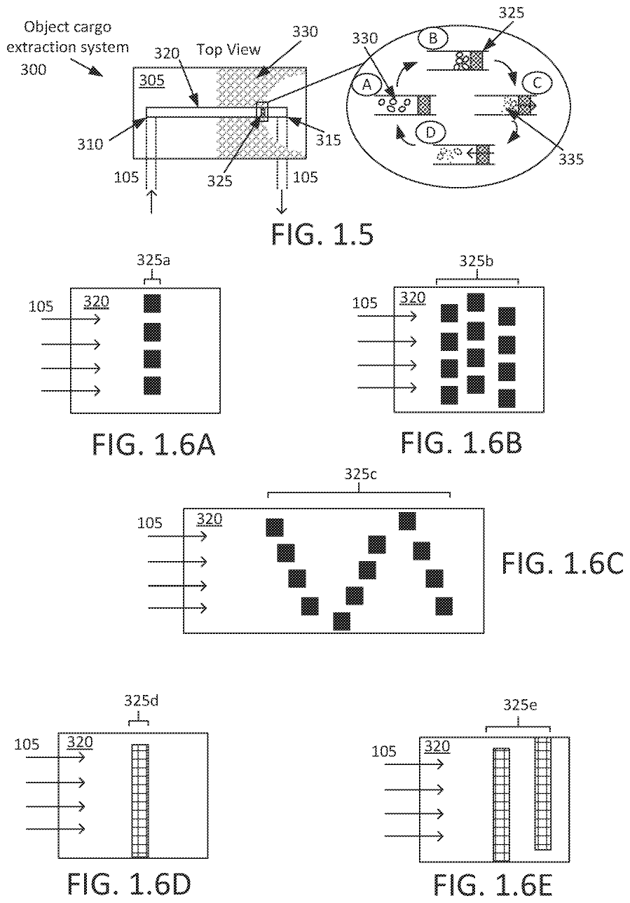

FIG. 1.1 illustrates an embodiment of the analysis system 100 of the present disclosure. The analysis system 100 includes a sample introduction system 200, an object cargo extraction system 300 and a detection system 400. While the analysis system 100 illustrates one of each of the sample introduction system 200, the object cargo extraction system 300 and the detection system 400, one or more of each can be used in parallel and/or serially depending on the desired results, design considerations and the like. Components such as tubing and valves can interconnect the various systems to transport fluid samples, conditioning fluids, and/or waste throughout the analysis system 100. Reference is often made to the various systems as being in “fluidic communication”, which means that components such as tubing and valves known in the art can connect the various systems, where the valves can include two or more options for flowing the fluids and/or introducing fluids in a forward mode or reverse mode. While there is much detail regarding the interconnection of the various systems not every conceivable variable is provided but one of skill would understand how to design and construct the interconnections.

In general, the sample introduction system 200 functions to introduce the sample (e.g., which may be acquired from a cell culture sample of the like) to the object cargo extraction system 300. The sample introduction system 200 can operate in-line or at-line (e.g., see FIGS. 1.3, 1.4, and 3.1A-3.1C), which are described in more detail herein and in Examples 1 and 2. In an aspect, the targeted cargo-containing objects are cells, extracellular vesicles, organelles, bacteria, viruses, synthetic membrane-bound particles, synthetic scaffolds to which cells are bound, or a combination thereof. The cargo of the targeted cargo-containing object corresponds to the particular targeted cargo-containing object. The cargo can include biomolecules such as proteins, peptides, nucleotides, DNA, RNA, sugars, proteases, growth factors, chemokines, cytokines, adhesion molecules, fatty acids, lipids, amines, co-factors, organic acids, polysaccharides, metabolites, and the like. In addition, the cargo can include secretome, metabolome, transcriptome, genome, lipidome, organelles, as well as other components found in cytoplasm of a cell or packaged in a synthetic cargo delivery object.

In general, the object cargo extraction system 300 functions to trap the targeted cargo-containing object(s) (e.g., cells) using a channel including one or more capture features, remove (e.g., lyse) the cargo from the targeted cargo-containing object(s), and flow the cargo to the detection system 400. A substantial portion, or all, of the debris (e.g., cell walls, viral capsid, viral envelope, membrane, and the like) from the cargo removal process (e.g., electrical lysis) does not flow to the detection system 400 due to the one or more capture features. In short, the one or more capture features are a “barrier” that prevents, or substantially prevents, the targeted cargo-containing object and the debris from lysing the targeted cargo-containing object from flow beyond the one or more capture features in the channel. In an aspect, the object cargo extraction system can include two or more channels operated in parallel or serially.

In general, the detection system 400 functions to detect the cargo of the targeted cargo-containing object. The detection system 400 can include an electrospray ionization device interfaced with a mass spectrometry system, an ion trapping system, an optical spectroscopy sensor, a Raman spectroscopy, a FTIR Spectrometer, a UV-VIS Spectrometer, a nuclear magnetic resonance spectroscopy, an electrochemical redox and/or impedance sensor, a mass cytometry system, or a flow cytometry system, or a combination thereof. In an aspect, the detection system 400 includes an electrospray ionization device interfaced with a mass spectrometry system, such as described in the Examples. In an aspect, two or more detection systems can be used, wherein the same or different types of detectors (e.g., mass spectrometry system or optical spectroscopy sensor) can be selected based on the type of cargo, type of detection desired, or the like.

In an aspect, the mass spectrometry system and the ion trapping system can include, but are not limited to, a time-of-flight (TOF) mass spectrometry system, an ion trap mass spectrometry system (IT-MS), a quadrapole (Q) mass spectrometry system, a magnetic sector mass spectrometry system, and an ion cyclotron resonance (ICR) mass spectrometry system, and combinations thereof. The mass spectrometry system and the ion trapping system can include an ion detector for recording the number of ions that are subjected to an arrival time or position in a mass spectrometry system, as is known by one skilled in the art. Ion detectors can include, for example, a microchannel plate multiplier detector, an electron multiplier detector, or a combination thereof. In addition, the mass spectrometry system includes vacuum system components and electric system components, as are known by one skilled in the art. In an aspect, the detection system 400 includes an electrospray ionization device interfaced with a mass spectrometry system, such as described in the Examples.

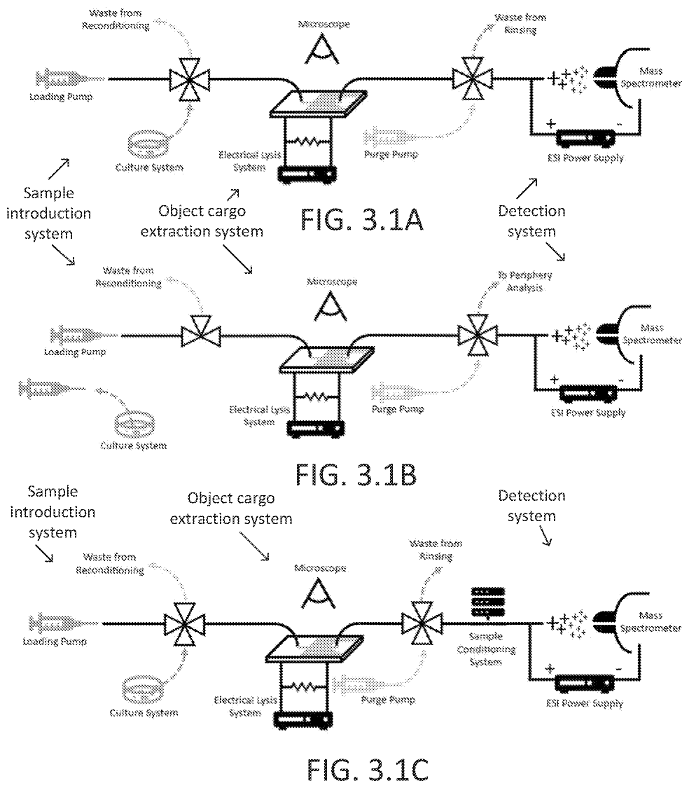

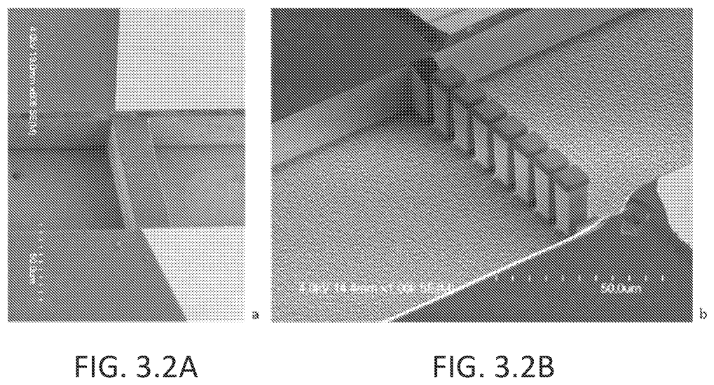

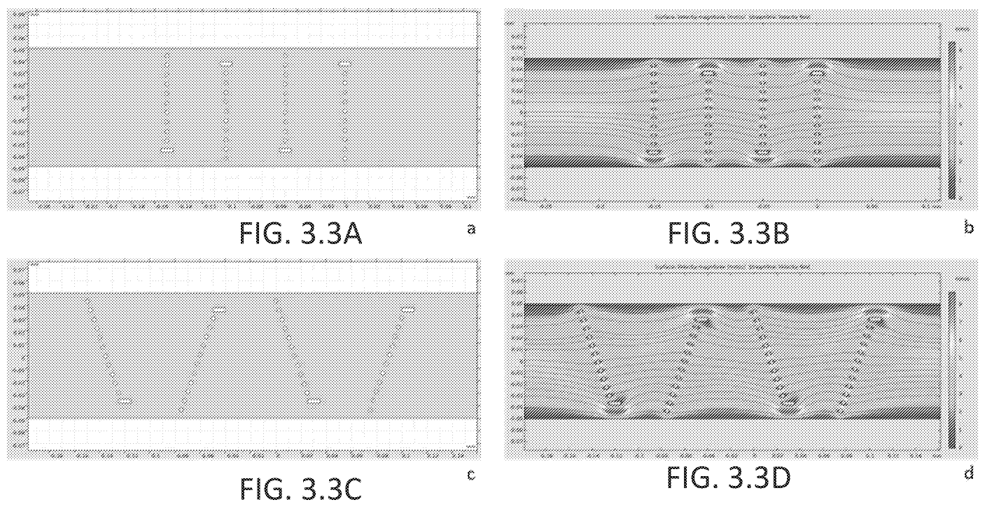

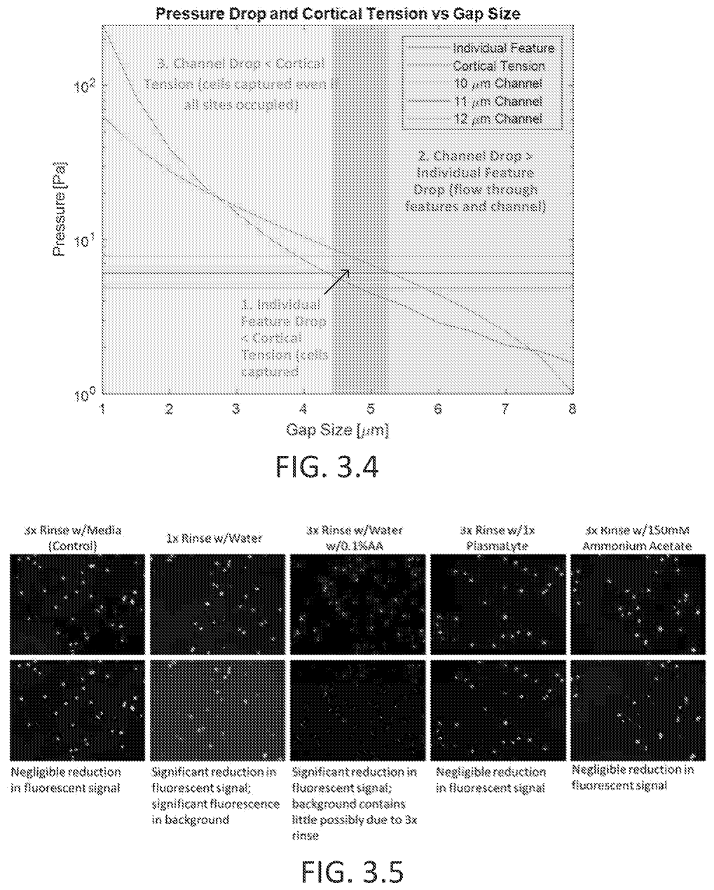

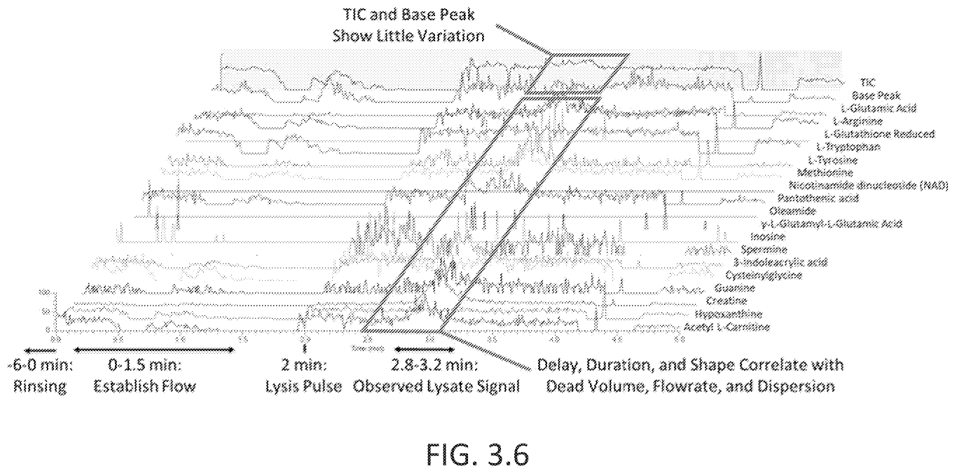

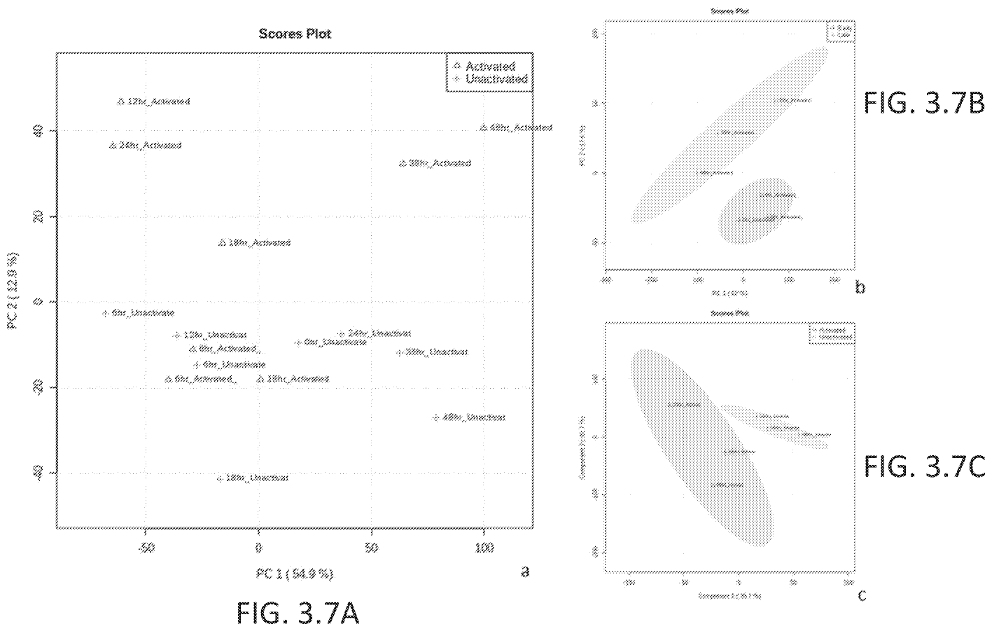

FIG. 1.2 illustrates an embodiment of the analysis system 100 that includes additional detail. Other embodiments of the analysis system are provided for in FIGS. 3.1A-3.1C. The arrows present in FIG. 1.2 and other figures indicates that the components are in fluidic communication. The analysis system 100 includes a sample introduction system 200 that is in fluid communication with a first flow valve 210. The first flow valve 210 can have multiple settings so that different components of the analysis system 100 are in fluidic communication, and each will be described herein. In a forward flow (105), the sample introduction system 200 is in fluidic communication with the object cargo extraction system 300 via the first flow valve (in a first setting). The sample can be flowed from the sample introduction system 200 to the object cargo extraction system 300 in the forward flow configuration. The targeted cargo-containing objects can be trapped and lysed in the object cargo extraction system 300 so that the cargo can be flowed to the detection system 400. The object cargo extraction system 300 is in fluidic communication with the detection system 400 via a second flow valve 310 (in a first setting). The detection system 400 is configured to analyze the cargo.

In an aspect, the sample including the targeted cargo-containing object can be rinsed in the object cargo extraction system 300. Once trapped by the one or more capture features, a conditioning fluid (e.g., selected to prevent osmotic lysis of the targeted cargo-containing objects) can be flowed across the targeted cargo-containing objects to rinse extra-media (e.g., extracellular) from the sample of targeted cargo-containing objects. The flow (105 to 115) of the rinse will flow through the second flow valve (in a second setting) to a waste collection device or a secondary analysis device. The secondary analysis device is optionally present and can include each of the analysis devices listed in respect to the detection system 400 as well as chromatography devices (e.g., HPLC), ion mobility spectrometer, optical spectroscopy sensor, a Raman spectroscopy, a FTIR Spectrometer, a UV-VIS Spectrometer, a nuclear magnetic resonance spectroscopy, an electrochemical redox and/or impedance sensor, a mass cytometry system, a flow cytometry system, antibody-antigen assays, or a combination thereof, and the like.

In an aspect, the analysis system 100 can operate in a reverse flow mode (110) to rinse out the object cargo extraction system 300. In short, the targeted cargo-containing objects are trapped by the one or more capture features and then the targeted cargo-containing objects are lysed. The cargo of the targeted cargo-containing objects can flow through the one or more capture features, while the debris from the targeted cargo-containing objects after lysing remains in the channel. As a result, the debris needs to be removed. The debris can be removed using the reverse flow mode. A purge pump 330 can pump a purge fluid (e.g., solutions containing DNase, lipase, trypsin, organic solvents, detergents, or a combination thereof to aid in removal of the debris) through the second flow valve 310 (in a third setting) into the channel of the object cargo extraction system 300, which washes away the debris. The purge fluid with the debris flows to the first flow valve 210 (in a second setting) to the first waste device 220.

In an optional aspect, the sample including the targeted cargo-containing object can flow (105 to 125) through the first flow valve 210 (in a fourth setting) to a conditioning system 340. The conditioning system 340 can be configured to remove unwanted components present in the fluid sample, introduce signal enhancing components, perform solid phase extraction, perform liquid-liquid extraction, perform electrophoretic separation, perform size exclusion, perform chromatographic separation, perform precipitation based separation, perform electrokinetic separation, perform magnetic separation, perform centrifugation, perform selective component vaporization, perform acoustic separation, perform thermophoresis, or a combination thereof, to produce a conditioned sample of conditioned targeted cargo-containing objects. The conditioned sample of conditioned targeted cargo-containing objects can then flow from the conditioning system 340 back to the first flow valve 210 (now in a fifth setting) to flow to the object cargo extraction system 300. Optionally, the conditioning system 340 can be placed between the sample introduction system 200 and the object cargo extraction system 300, albeit such a set up may necessitate inclusion of two conditioning systems and/or additional flow routes.

In an optional aspect, the cargo can be flowed (105 to 120) from the object extraction system 300 to the conditioning system 340. The conditioning system 340 can be configured to remove unwanted components present in the fluid sample, retain target cargo and remove non-target cargo, introducing detector signal selectivity and sensitivity enhancing components, introducing biochemical standards, isotope-coded affinity tags, tandem-mass spectrometry tags, perform solid phase extraction, perform liquid-liquid extraction, perform electrophoretic separation, perform size exclusion, perform chromatographic separation, perform precipitation based separation, perform electrokinetic separation, perform magnetic separation, perform centrifugation, perform selective component vaporization, perform acoustic separation, perform thermophoresis, or a combination thereof. The conditioned cargo can then flow (120) from the conditioning system 340 to the detection system 400. Optionally, the conditioning system 340 can be placed between the sample introduction system 200 and the object cargo extraction system 300, albeit such a set up may necessitate inclusion of two conditioning systems and/or additional flow routes. While the conditioning system 340 have been described specifically above, the following provides some additional features of the conditioning system. The conditioning system can be configured to condition the fluid sample (e.g., conditioned cells) and/or the cargo (e.g., those collected from the targeted cargo-containing objects) to produce a conditioned sample (e.g., condition cells) or conditioned cargo. The conditioning can include removing unwanted components present in the fluid sample (e.g., remove or reduce the amount of components that can interfere with detection of the desired signal such as salts which can inhibit mass spectrometry signals), retaining relatively larger components such as biomolecules as compared to smaller components (e.g., small organic molecules), and introducing signal enhancing components (e.g., 3-nitrobenzyle alcohol (m-NBA), organic solvents, organic acids, inorganic acids, volatile salts, ammonium acetate, biological standards, conjugated antibodies, fluorescent dyes, molecular tags, or a combination thereof), which may lower the detection limit, improve signal to noise ratio, shift charge distributions to mitigate effects of unwanted components, and the like. The conditioning fluid for the sample can be different than the condition fluid for the cargo.

In regard to mass spectrometry, the conditioning fluid may include organic acids such as acetic acid, trifluoroacetic acid (TFA), formic acid, etc. (e.g., 0% to 100%), which can aid in protonation of biomolecules, for example. The conditioning fluid can also include one or more of the following: ammonium acetate (e.g., 0% to 100%), m-NBA (e.g., 0% to 100%), propylene carbonate (e.g., 0% to 100%), ethylene carbonate (e.g., 0% to 100%), sulofane (e.g., 0% to 100%), and organic solvents such as methanol, acetonitrile, isopropyl alcohol (IPA), chloroform, acetone, N-methyl-2-pyrrolidone (NMP), etc. (e.g., 0% to 100%), chemical standards (i.e. to reduce instrument drift and enable quantitative analysis), and a combination thereof. Ammonium acetate can increase the acidity in the electrospray plume, enhance protonation, and/or reduce formation of salt adducts. M-NBA can increase the charge state in non-denaturing fluid samples. Methanol, and other organic solvents, can be used to selectively remove biomolecules with preferential solubility in the organic solvent and also to shift the charge state distribution of larger biomolecules like proteins through denaturing and unfolding effects on the molecule. In the case of in-line HPLC or direct ESI-MS analysis, chemical standards can be added to the conditioning flow to help with quantification of sample concentration and accurate mass identification.

In an aspect, the conditioning system can include two or more flow channels and one or more selectively permeable membrane, where the selectively permeable membranes are adjacent one or more of the flow channels. In an aspect, the conditioning system includes a first flow channel having a first flow channel entrance and a first flow channel exit. In addition, the conditioning system includes a second flow channel having a second flow channel entrance and a second flow channel exit. The first flow channel and the second flow channel can be separated from one another by a selectively permeable membrane. As fluid flow through the flow channels, the fluids in each flow channel are in fluidic communication with the selectively permeable membrane.

The material defining the first flow channel and the second flow channel can be made of a polymer, ceramic, glass, silicon, plastic, or polyamide, metal, or PDMS. Each flow channel can have a height of about 1 μm to 1 mm and a width of about 1 μm to 10 mm. The first flow channel and the second flow channel can be adjacent the selectively permeable membrane for a length of about 100 μm to 100 mm.

The selectively permeable membrane functions to separate unwanted components in the fluid sample or in the cargo fluid from those of interest and/or to cause the introduction of components to the fluid sample or cargo fluid to enhance detectability. The selectively permeable membrane can be made of material such as aluminum oxide (anodized porous alumina), polymers (e.g., track etch membranes), cellulose, and zeolite, porous metal (e.g., nanoporous copper), porous graphene and graphene oxide. The selectively permeable membrane can be made of or coated with a material that aids the formation of conditioned fluid sample. For example, the selectively permeable membrane can be made of or coated with a hydrophobic material/hydrophilic material, lipophilic material/lipophobic material, inert material, decorated with selectively (positively or negatively) charged chemical compounds, electrically conducting, semiconducting or insulating material, and combinations thereof. The selectively permeable membrane can have a porosity of about 5% to 95%. The selectively permeable membrane can have a thickness of about 1 nm to 10 μm, a length of about 10 μm to 50 mm, and a width of about 10 μm to 10 mm.

In an aspect, the conditioning system can be configured to flow the fluid sample flow through the first flow channel from the first flow channel entrance to the first flow channel exit and be in fluid communication with the selectively permeable membrane. In addition, the conditioning system can be configured to flow a conditioning fluid through the second flow channel from the second flow channel entrance and the second flow channel exit and be in fluid communication with the selectively permeable membrane, where the sample fluid and the conditioning fluid are in communication through the selectively permeable membrane. Although here as well in other embodiments the flow of the fluid sample and the conditioning fluid are in the same direction, the fluid flow can be change so the two flow counter to one another or across one another.

FIGS. 1.3 and 1.4 illustrate in-line operation of the analysis system 100 and at-line operation of the analysis system 100, respectively. These embodiments are also described in the Examples. The sample introduction system 200 in FIG. 1.3 includes a loading pump 215 and set up in an in-line operation configuration. The loading pump 215 can acquire the sample from a sample container 220 via the first flow valve 210 (in a third position). The loading pump 215 can draw the sample from the sample container 220 through the first valve 210 to the sample introduction system 200. Once the loading pump 215 includes the sample, the first valve 210 can be changed to the first setting so that the sample introduction system 200 is in fluidic communication with the object cargo extraction system 300 and process accordingly.

The sample introduction system 200 in FIG. 1.4 includes a loading pump 215 and set up in an at-line operation configuration. The loading pump 215 is removable from the sample introduction system 200 and separately collects the sample (140). The loading pump 215 is then interfaced with the sample introduction system 200. Once the loading pump 215 includes the sample, the first valve 210 can be put into the first setting so that the sample introduction system 200 is in fluidic communication with the object cargo extraction system 300 and process accordingly.

The loading pump can include a syringe pump, piezoelectric pump, peristaltic pump, centrifugal pump, positive displacement pump, rotary pump, diaphragm pump, or capillary suction pump. For example, a KDS Scientific Legato 270 syringe pump can cause a fluid sample of about 1 μL to 100 mL to flow through the analysis system. The loading pump can be operated manually and/or by a computer system. The loading pump can include an extraction element such as a needle, tube, capillary tube, straw, porous flow structure, or the like. The extraction element can be dimensionally configured to extract the fluid sample. The extraction element can be dimensionally configured to extract the fluid sample comprising one or more intact cells. For example, the inner diameter of the extraction element can be about 10 μm to 100 μm for an intact cell. In addition, the extraction element can include a filter or other components to limit what is extracted in the fluid sample.

Having described the overall analysis system and components of the analysis system, additional details regarding the object cargo extraction system are provided. FIG. 1.5 illustrates a top view of an embodiment of the part of the object cargo extraction system 300. The object cargo extraction system 300 includes a structure 305 having an inlet 310 and an outlet 315 for a channel 320 (e.g., the structure can have a plurality on inlets and/or outlets). While the inlet and outlet are positioned at opposing ends, other configurations are contemplated. Also, channel 320 is depicted as straight, but the channel can be curved, serpentine, “S” shaped, “U” shaped, or have another geometry. One or more capture features 325 are present in the channel 320, where the one or more capture features can be located at any point along the channel 320 or along the entire length of the channel 320. Electrodes 330 for lysing the targeted cargo-containing objects are present adjacent to the channel 320. The electrodes 330 can be in a specific area along the channel or along the entire length of the channel 320. The channel 320 can have a length of about 10 μm to 10 cm, a width of about 100 nm to 1 mm, and a height of about 10 nm to 1 mm. The channel 320 can have a height of about 10 nm to 500 μm (or more for larger targeted cargo-containing objects), while the channel can have a width of about 10 nm to 500 μm (or more for larger targeted cargo-containing objects), where the height and/or width can be selected based on the dimensions of the targeted cargo-containing objects. The channel 320 can have a volume of about 0.01 cubic μm to 1e¹⁰ cubic μm. The dimensions of the channel 320 can be designed based on the type of targeted cargo-containing objects, for example, the dimensions are larger for cells and smaller for organelles. In an aspect, the dimensions (e.g., height and/or width) of the channel can range from 10 s of nm (e.g., about 10 to 100 nm or about 10 to 50 nm) for small extracellular vesicles and viruses to 100s of nm or single μm (e.g., about 100 nm to 1 μm or about 100 nm to 500 nm) for bacteria and organelles to single μm for cells to 100s of μm for synthetic scaffolds for 3D cell culture (e.g., about 1 to 50 μm or about 10 to 500 μm). Similarly, the dimensions of the capture features 325 are selected based on the type of targeted cargo-containing objects. The dimensions and design of the channel and the capture features can be dependent on one another and selected based on the type of targeted cargo-containing objects.

The right side of FIG. 1.5 illustrates the introduction (A) of the targeted cargo-containing objects 330 to the channel 320. The targeted cargo-containing objects 330 are trapped (B) adjacent the capture features 325. The targeted cargo-containing objects 330 can be lysed (C) so that the cargo flow past the capture features 325 while the debris 335 is prevented, or substantially prevented, from flowing past the capture features 325. The debris 335 can be removed from the channel in the reverse flow mode (as described above and herein).

As described above and in reference to FIG. 1.5, the targeted cargo-containing objects can be lysed using an electrical pulse(s) from the electrodes (330). One or more electrical pulses can be applied to the trapped targeted cargo-containing objects. The pulse(s) can be applied such that the membrane of the targeted cargo-containing cells is irreversibly disrupted by exceeding the transmembrane potential for onset of the formation of pores within the membrane. For example, for mammalian cells this threshold is commonly cited as being approximately 1V corresponding to an electric field strength of 1 kV/cm assuming a 10 μm cell. For DC application of the electrical pulse, the pulse duration can be applied such that membrane poration has a duration sufficient to become irreversible, typically in the range of 100s of ns to seconds. For AC application of the electrical pulse, the frequency can be set to ensure the transmembrane potential is exceeded for sufficient duration each waveform cycle for irreversible poration, typically in the range of Hz to MHz. The pulse configuration (e.g., electrical field strength, waveform, duration, etc.) can be modified dependent on the membrane-structure to be disrupted. In an aspect, the applied electric field strength could be about 0.5 kV/cm to 50 kV/cm or about 1 kV/cm to 20 kV/cm or about 5 kV/cm to 10 kV/cm; the period of the square wave pulse of about 0.01 ms to 1 s or about 1 ms to 500 ms or about 10 ms to 100 ms with variable duty cycle (duration of the DC pulse relative to the period of the square wave) in the range of about 1% to 99% or about 20% to 80%, or about 40% to 60%. In a particular aspect, an electric field strength of about 6 kV/cm can be applied between the electrodes with square wave DC pulse of about 5 ms duration and about 10 ms period; having about 1000 pulses applied. In another aspect, an electric field strength of about 20 kV/cm can be applied between the electrodes with square wave DC pulse of about 100 ms duration and about 200 ms period; having about 300 pulses applied. In an aspect, the electrodes (e.g., the object cargo extraction system) are electrically decoupled from other components of the analysis system. For example, the electrodes are electrically decoupled from the electrospray system. In an aspect, the decoupling can be accomplished using a circuit described in the Examples.

The object cargo extraction system 300 includes a channel including one or more capture features. The capture features function to trap the targeted cargo-containing objects so that the targeted cargo-containing objects can be lysed. In an aspect, the capture features are a physical barrier that traps the targeted cargo-containing objects. The capture features can be made of a polymeric material, silicon, glass, and metal, ceramics, etc. The capture features can have various shapes such as pillars having a circular, square, rectangle, concave or convex open cavities, airfoil, etc. cross-section or a semipermeable membrane (e.g., permeable to the cargo). The capture features can be symmetrical or asymmetrical, where an asymmetric design in the direction of flow can facilitate capture during targeted cargo-containing object loading while facilitating backflow of object debris following electrical lysis and analysis. The number and/or dimensions of the capture features depend at least upon the dimensions of the channel and the dimensions of the targeted cargo-containing objects. The plurality of capture features are designed and positioned in the channel to allow for fluid to pass through spaces between the capture features and the space between the capture features and the channel walls, the targeted cargo-containing object does not flow past the capture features. In particular, capture features and spacing between the capture features have dimensions based on the dimensions of the targeted cargo-containing object and the channel, where the dimension of the spacing between the capture features relative and/or wall of the channel to the dimensions of the targeted cargo-containing object is based on not allowing the targeted cargo-containing object to flow past the one or more capture features while allowing the cargo (e.g., target components, solvated components, or both) to flow past the one or more capture features. The spaces between capture features and capture features and the channel walls can be uniform but can do not have to be uniform as long as the capture features function as intended. Additional details regarding design of the capture features are provided in the Examples.

In a particular aspect, the one or more capture features include a plurality of pillars. The spacing between the pillars and the pillars and the channel walls is such that the targeted cargo-containing object does not flow past the pillars but the cargo can flow through the pillars. In one aspect, the plurality of pillars includes two or more rows of pillars, where the spacing between the rows of pillars, the spacing between the pillars in each row, the spacing between the pillars and the channel walls, or a combination of these is such that the targeted cargo-containing object does not flow past the one or more capture features, but the cargo can flow through the pillars. In another aspect, the plurality of pillars includes two or more groups of pillars (e.g., a diagonal row, checkerboard layout, etc.), where the spacing between the groups of pillars, the spacing between the pillars in each group, the spacing between the pillars and the channel walls, or a combination of these is such that the targeted cargo-containing object does not flow past the one or more capture features, but the cargo can flow through the pillars.

As described herein, the plurality of capture features are designed and positioned in the channel to allow for fluid to pass through spaces between the capture features and the space between the capture features and the channel walls, and the targeted cargo-containing object does not flow past the capture features. Other considerations regarding the capture features include that the capture features should are structurally sound throughout prolonged operation while under various pressure changes. In an aspect, the capture features can be designed to limit the pressure drop across the capture features such that the backpressure experienced by the targeted cargo-containing objects does not lead to the objects pressing into or through the capture features. In an aspect, the axial length should generally be minimized to reduce pressure drop across the feature while maintaining structural integrity, while the width of each capture features should generally be minimized to maximize the total flow area between the capture features while maintaining structural integrity.

The capture features can be designed (e.g., dimensions, materials, spacing, etc.) so that the pressure drop across an individual capture feature is less than the critical pressure for the targeted cargo-containing object to pass through. In an aspect, a set of capture features can include a larger gap such that the total pressure drop across the set of features remains below the critical pressure even when all capture sites are occupied (See FIG. 3.4). In this way, multiple sets of capture features can be used to trap the targeted cargo-containing objects while also maintaining pressure thresholds for operation.

In an aspect, the size of the space (e.g., distance) between capture features and capture features and the channel walls should be smaller than the targeted cargo-containing objects or small enough to prevent the targeted cargo-containing features from deforming to squeeze past the capture features. The selection of the size of the space between capture features and capture features and the wall channels is based on the smallest anticipated targeted cargo-containing objects to be retained or selected to capture. In general, the space between capture features and captures features and the channel walls can be about 10s of nanometers (e.g., about 10 to 250 nm, about 10 to 100 nm, or about 10 to 50 nm) for viruses and extracellular vesicles. The space between capture features and captures features and the channel walls can be about 100s of nanometers (e.g., about 10 to 500 nm, about 10 to 250 nm, about 10 to 100 nm, or about 10 to 50 nm) for bacteria and organelles. The space between capture features and captures features and the channel walls can be about micron (e.g., about 0.1 to 100 μm, about 1 to 50 μm, or about 1 to 10 μm) for cells. The space between capture features and captures features and the channel walls can be about 100s micron (e.g., about 100 to 200 μm, about 100 to 300 μm, about 100 to 500 μm) for synthetic scaffolds for 3D cell culture.

In another aspect, the one or more capture features includes one or more permeable membranes (such as those described above and herein), where the targeted cargo-containing objects do not flow past the one or more permeable membranes. The permeable membrane can include anodized porous alumina, etched silicon, perforated polymer, plastic or metal films, cellulose sheet, or a packed bed of various materials. In an aspect, the largest pore opening should be smaller than the targeted cargo-containing object or smaller than the targeted cargo-containing object can contort themselves (e.g., the opening that the targeted cargo-containing object can fit through may be smaller than the actual diameter of the targeted cargo-containing object), while also the average porosity should be maximized to limit flow restriction across the membrane while maintaining structural integrity.

Now having described the capture features generally, FIGS. 1.6A-1.6E illustrate representative embodiments of the capture features. While these representative configurations are provided, other configurations are contemplated. FIG. 1.6A illustrates a top view a single row of capture features 325a. FIG. 1.6B is a top view that illustrates three rows of capture features 325b, where the capture features of the rows are offset, which can be done to prevent pressure to build up as well as to prevent targeted cargo-containing objects from passing through the capture features 325b. FIG. 1.6C is a top view that illustrates three group of capture features in a diagonal or zig-zag configuration. FIG. 1.6D is a top view that illustrates the capture feature as a permeable membrane 325d, where a space is included between the channel wall and the permeable membrane to ensure flow of the fluid is maintained so the proper pressure is maintained. FIG. 1.6E is a top view that illustrates two permeable membranes 325e, where multiple spaces are provided to ensure proper pressure is maintained. While these figures show a top view, spacing between the capture features and/or the walls of the channel can be present on any side and can be varied to accomplish the desired trapping of the targeted cargo-containing objects and flow of fluid. The capture features can be designed for bulk capture of targeted cargo-containing objects in the flow (e.g., indiscriminate filter) or for capture of individual targeted cargo-containing objects (e.g., single cell capture at individual capture sites). The number and design of capture features can be such that a de facto targeted cargo-containing object count is obtained following loading of the targeted cargo-containing objects based on capture efficiency. The capture features designs can be such that there is selective capture of some targeted cargo-containing objects while other targeted cargo-containing objects are not retained. Such selective capture could be used to assess different subsets of the sample (e.g., different types of cells, different cell phenotypes, different types of extracellular vesicles, etc.). The capture features could be arranged such that selective retention enables sorting with different subsets of targeted cargo-containing objects being captured in different regions of the channel or different channels such that analysis can be sequentially performed on the subsets. The capture features could be coated with one or more molecular recognition layers which are selectively “sticky” (i.e., preferential chemically binding) to the specific targeted cargo-containing objects. Additional details regarding capture feature dimension and spacing are provided in the Examples.

While various embodiments of the present disclosure have been provided, the following aspects provide additional details and combinations of embodiments of the present disclosure.

Aspect 1. An analysis system comprising:
a sample introduction system configured to deliver a sample, wherein the sample introduction system includes an inlet and an outlet (e.g., two or more inlets and/or outlets are optional), wherein the sample includes one or more targeted cargo-containing objects (optionally, one or more additional sample introduction system can be included);
an object cargo extraction system, wherein the object cargo extraction system includes an inlet and an outlet (e.g., two or more inlets and/or outlets are optional), wherein a channel (e.g., two or more channels are optional) includes one or more capture features positioned between the inlet and the outlet, wherein the one or more capture features are configured to trap the targeted cargo-containing objects in the sample as the sample flows through the channel from the inlet to the outlet, wherein electrodes are positioned adjacent to an area of the channel, wherein the electrodes are configured for electrical lysis of targeted cargo-containing objects present in the area of the channel, wherein the inlet of the object cargo extraction system is in fluidic communication with the outlet of the sample introduction system (optionally one or more additional object extraction systems can be included and/or one or more of these can be in communication with one another or with the one or more sample introduction systems); and
a detection system, wherein the object cargo extraction system is in fluidic communication with the detection system, wherein the detection system is configured to detect cargo released from the targeted cargo-containing objects (optionally one or more detection systems can be included and in communication with the one or more object cargo extraction systems).

Aspect 2. The system of any of the aspects provided, wherein the one or more capture features comprises a plurality of pillars, wherein the spacing between the pillars and the pillars and the channel walls is such that the targeted cargo-containing object does not flow past the pillars.

Aspect 3. The system of any of the aspects provided, wherein the plurality of pillars comprises two or more rows of pillars, wherein the spacing between the rows of pillars, the spacing between the pillars in each row, the spacing between the pillars and the channel walls, or a combination of these is such that the targeted cargo-containing object does not flow past the one or more capture features.

Aspect 4. The system of any of the aspects provided, wherein the plurality of pillars comprises two or more groups of pillars, wherein the spacing between the groups of pillars, the spacing between the pillars in each group, the spacing between the pillars and the channel walls, or a combination of these is such that the targeted cargo-containing object does not flow past the one or more capture features.

Aspect 5. The system of any of the aspects provided, wherein the one or more capture features comprises one or more permeable membranes, where the targeted cargo-containing objects do not flow past the one or more permeable membranes.

Aspect 6. The system of any of the aspects provided, wherein the one or more capture features and spacing between the features and the walls of the channel have dimensions based on the dimensions of the targeted cargo-containing object, wherein the dimension of the spacing between the capture features and the channel walls relative to the dimensions of the targeted cargo-containing object is based on not allowing the targeted cargo-containing object to flow past the one or more capture features while allowing the cargo released from the targeted cargo-containing objects to flow past the one or more capture features.

Aspect 7. The system of any of the aspects provided, wherein the channel has a length of about 10 μm to 10 cm, a width of about 100 nm to 1 mm, and a height of about 10 nm to 1 mm.

Aspect 8. The system of any of the aspects provided, wherein the sample introduction system is in fluidic communication with a first flow valve, wherein a first setting of the first flow valve is configured to be in communication with the inlet of the object cargo extraction system, wherein the sample in the sample introduction system is flowed through the first valve in the first setting into the object cargo extraction system.

Aspect 9. The system of any of the aspects provided, wherein the sample introduction system is in fluidic communication with a first flow valve, wherein a first setting of the first flow valve is configured to be in communication with the inlet of the object cargo extraction system, wherein a second setting of the first flow valve is configured to be in communication with a structure including the sample, wherein when the first flow valve is in the second setting, the sample introduction system is configured to withdraw the sample from the structure, wherein after withdrawal of the sample, the first flow valve is adjusted to the first setting and the sample is flowed through the first flow valve into the object cargo extraction system.

Aspect 10. The system of any of the aspects provided, wherein the detection system is an electrospray ionization mass spectrometry system, ionization and sensing device such ion mobility spectrometer, optical spectroscopy sensor, Raman spectroscopy, FTIR Spectrometer, UV-VIS Spectrometer, nuclear magnetic resonance spectroscopy, electrochemical redox and/or impedance sensor, mass cytometry system, or flow cytometry system.

Aspect 11. The system of any of the aspects provided, wherein the electrospray mass spectrometry system includes an electrospray ionization device, wherein the electrospray ionization device and the electrodes of the object cargo extraction system are electrically decoupled.

Aspect 12. The system of any of the aspects provided, wherein the object cargo extraction system is in fluidic communication with a first waste receiving device in a reverse flow configuration, wherein the object cargo extraction system is in fluidic communication with a purge pump in the reverse flow configuration, wherein a purge fluid is flowed through the object cargo extraction system to the first waste receiving device in the reverse flow configuration.

Aspect 13. The system of any of the aspects provided, wherein the first waste receiving device in fluidic communication with the inlet of the object cargo extraction system when a first flow valve is in a third setting, wherein the first flow valve is positioned between the sample introduction system and the object cargo extraction system,
wherein the purge pump is in fluidic communication with the outlet of the object cargo extraction system when a second flow valve is in a third setting, wherein second flow valve is positioned between the outlet of the object cargo extraction system and the detection system,
wherein the purge pump is in fluidic communication with the first waste receiving device when the first flow valve is in the third setting and the second flow valve is in the third setting such that the purge pump is configured to flow the purge fluid through the object cargo extraction system to the first waste receiving device.

Aspect 14. The system of any of the aspects provided, further comprising a second waste receiving device in fluidic communication with the object cargo extraction system when a second flow valve is in a second setting, wherein the second flow valve is positioned between the object cargo extraction system and the second waste receiving device.

Aspect 15. The system of any of the aspects provided, further comprising a secondary analysis system in fluidic communication with the object cargo extraction system when a second flow valve is in a second setting, wherein the second flow valve is positioned between the object cargo extraction system and the secondary analysis system.

Aspect 16. The system of any of the aspects provided, further comprising a conditioning system (e.g., two or more conditioning systems are optional) positioned between the object cargo extraction system and the detection system, wherein the conditioning system is configured to remove unwanted components present in the fluid sample, retain target components and remove non-target components, introduce signal enhancing components, perform solid phase extraction, perform liquid-liquid extraction, perform electrophoretic separation, perform size exclusion, perform chromatographic separation, perform precipitation based separation, perform electrokinetic separation, perform magnetic separation, perform centrifugation, perform selective component vaporization, perform acoustic separation, perform thermophoresis, or a combination thereof.

Aspect 17. The system of any of the aspects provided, further comprising a conditioning system (e.g., two or more conditioning systems are optional) positioned between the sample introduction system and the object cargo extraction system, wherein the conditioning system is configured to remove unwanted components present in the fluid sample, introduce detection signal enhancing components, perform solid phase extraction, perform liquid-liquid extraction, perform electrophoretic separation, perform size exclusion, perform chromatographic separation, perform precipitation based separation, perform electrokinetic separation, perform magnetic separation, perform centrifugation, perform selective component volatilization, perform acoustic separation, perform thermophoresis, or a combination thereof.

Aspect 18. The system of any of the aspects provided, further comprising a second object cargo extraction system, wherein the second object cargo extraction system includes an inlet and an outlet, wherein the channel includes one or more capture features positioned between the inlet and the outlet, wherein the one or more capture features are configured to trap the targeted cargo-containing objects in the sample as the sample flows through the channel from the inlet to the outlet, wherein electrodes are positioned adjacent to an area of the channel, wherein the electrodes are configured for electrical lysis of targeted cargo-containing objects present in the area of the channel, wherein the inlet of the object cargo extraction system is in fluidic communication with the outlet of the sample introduction system.

Aspect 19. The system of any of the aspects provided, wherein the object cargo extraction system and the second object cargo extraction system are configured to operate in parallel or in series.

Aspect 20. The system of any of the aspects provided, wherein the object cargo extraction system includes an optical access area to monitor the sample in the channel.

Aspect 21. The system of any of the aspects provided, wherein the targeted cargo-containing object are cells, extracellular vesicles, organelles, bacteria, viruses, synthetic membrane-bound particles, synthetic scaffolds to which cells are bound, or a combination thereof.

Aspect 22. The system of any of the aspects provided, wherein the targeted cargo-containing object are cells.

Aspect 23. A method of analysis, comprising:
introducing a sample to an object cargo extraction system, wherein the sample includes extra-object media and targeted cargo-containing objects, wherein the object cargo extraction system includes a channel, an inlet, an outlet, and a one or more capture features positioned between the inlet and the outlet, wherein the one or more capture features trap the targeted cargo-containing objects in an area of the channel, wherein the object cargo extraction system includes electrodes positioned adjacent the channel, wherein the electrodes are configured for electrical lysis of targeted cargo-containing objects present in the area of the channel;
applying one or more electrical pulse across the electrodes to lyse the targeted cargo-containing objects, wherein the cargo present in the targeted cargo-containing objects pass through the one or more capture features, wherein other debris from lysing the targeted cargo-containing object do not pass through the one or more capture features;
flowing the cargo out of the outlet to a detection system, wherein the detection system is configured to identify the one or more of the cargo.

Aspect 24. The method of any of the aspects provided, further comprising: prior to lysing the targeted cargo-containing objects, removing extra-object media from the channel when the targeted cargo-containing objects are trapped by the one or more capture features using a liquid buffer that does not induce spontaneous lysis of the targeted cargo-containing objects, wherein the extra-object media pass through the one or more capture features.

Aspect 25. The method of any of the aspects provided, wherein the extra-object media is removed from the object cargo extraction system.

Aspect 26. The method of any of the aspects provided, wherein the extra-object media is flowed to a secondary analysis system.

Aspect 27. The method of any of the aspects provided, wherein the extra-object media is flowed to a second waste receiving device

Aspect 28. The method of any of the aspects provided, wherein the extra-object media is flowed out of the outlet of the object extraction system to a detection system, wherein the detection system is configured to identify the one or more of the components of the extra-object media.

Aspect 29. The method of any of the aspects provided, wherein after the components flow out of the outlet of the object cargo extraction system and prior to flowing into the detection system, the method includes conditioning the components to form conditioned components, wherein the condition components are flowed into the detection system.

Aspect 30. The method of any of the aspects provided, wherein conditioning includes removing unwanted components present in the fluid sample, retaining target components and removing non-target components, introducing detector signal selectivity and sensitivity enhancing components, introducing biochemical standards, isotope-coded affinity tags, tandem-mass spectrometry tags, or a combination thereof.

Aspect 31. The method of any of the aspects provided, wherein the signal enhancing components is selected from 3-nitrobenzyle alcohol (m-NBA), organic solvents, organic acids, inorganic acids, volatile salts, ammonium acetate, biological standards, conjugated antibodies, fluorescent dyes, molecular tags, or a combination thereof.

Aspect 32. The method of any of the aspects provided, wherein prior to flowing into the object cargo extraction system, the method includes conditioning the targeted cargo-containing objects to form conditioned targeted cargo-containing objects, wherein the conditioned targeted cargo-containing objects are flowed into the object cargo extraction system.

Aspect 33. The method of any of the aspects provided, wherein the detection system is an electrospray ionization mass spectrometry system, ionization and sensing device such ion mobility spectrometer, optical spectroscopy sensor, Raman spectroscopy, FTIR Spectrometer, UV-VIS Spectrometer, nuclear magnetic resonance spectroscopy, electrochemical redox and/or impedance sensor, mass cytometry system, or flow cytometry system.

Aspect 34. The method of any of the aspects provided, wherein the electrospray ionization device and the electrodes of the object cargo extraction system are electrically decoupled.

Aspect 35. The method of any of the aspects provided, wherein the sample is drawn from a structure containing the sample using suction pumping, wherein the sample passes through a first flow valve into a loading pump, then the first flow valve is re-directed to flow the sample into the inlet of the object cargo extraction system.

Aspect 36. The method of any of the aspects provided, wherein after the lysing and the cargo from the targeted cargo-containing object is flowed to the detection system, wherein the debris from the targeted cargo-containing object remaining in the channel are removed from the channel by flowing a purge fluid into the outlet of the channel, through the channel, out of the inlet, and then to a first waste receiving device.

Aspect 37. The method of any of the aspects provided, wherein the extra-object media are components from the sample not including the targeted cargo-containing object.

Aspect 38. The method of any of the aspects provided, wherein the targeted cargo-containing object are cells, extracellular vesicles, organelles, bacteria, viruses, synthetic membrane-bound particles, synthetic scaffolds to which cells are bound, or a combination thereof.

Aspect 39. The method of any of the aspects provided, wherein the targeted cargo-containing object are cells.

Aspect 40. The method of any of the aspects provided, wherein the targeted cargo-containing objects have cell walls, viral capsid, viral envelope, or membrane, and the cargos are within the cell walls, viral capsid, viral envelope, or membrane.

Aspect 41. The method of any of the aspects provided, wherein the debris comprises cell walls, viral capsid, viral envelope, membrane, or combinations thereof.

### EXAMPLES

Now having described the embodiments of the disclosure, in general, the examples describe some additional embodiments. While embodiments of the present disclosure are described in connection with the example and the corresponding text and figures, there is no intent to limit embodiments of the disclosure to these descriptions. On the contrary, the intent is to cover all alternatives, modifications, and equivalents included within the spirit and scope of embodiments of the present disclosure.

### Example 1

Real-time, advanced diagnostics of the biochemical state within cells remains a significant challenge for research and development, production, and application of cell-based therapies. The fundamental biochemical processes and mechanisms of action of such advanced therapies are still largely unknown, including the critical quality attributes that correlate to therapeutic function, performance, and potency and the critical process parameters that impact quality throughout cell therapy manufacturing. Example 1 provides for an integrated microfluidic platform for in-line analysis of a small number of cells via direct infusion nano-electrospray ionization mass spectrometry. An aspect of this platform is a microfabricated cell processing device that prepares cells from limited sample volumes removed directly from cell culture systems. The sample-to-analysis workflow overcomes the labor intensive, time-consuming, and destructive nature of existing mass spectrometry approaches for analysis of cells. By providing rapid, high-throughput analyses of the intracellular state, this platform enables untargeted discovery of critical quality attributes and their real-time, in-process monitoring.

Utilizing cells for therapeutic applications is revolutionizing medicine by providing cures for previously incurable diseases^{1,2} and treatment of previously irreparable injuries.³ These treatments, which rely on cells as either a drug production mechanism (e.g., monoclonal antibodies secreted from cells) or are directly used as the therapy (e.g., cell therapies), present unique development and biomanufacturing challenges compared to traditional small molecule pharmaceuticals. Rather than controlling the synthesis of a limited number of chemicals, cell-based therapies require not only characterization of the cell state, but also control of the complex and dynamic cell culture system as a whole.^{4,5} This presents a challenge for in-process control as existing real-time monitoring capabilities for biomanufacturing have been traditionally confined to indirect measures of culture viability such as temperature, pH, and dissolved gas.⁶ Direct monitoring of biochemicals in the extracellular environment (e.g., cell media) via various spectroscopic methods has vastly improved insight into bioprocess systems. These methods, however, are largely constrained to monitoring a limited number of the highest abundance species (e.g., lactose and glutamine) in culture media.^{7,8} As such, they provide little information as to the actual state of manufactured cells, effectively measuring time delayed and spatially averaged effects of cell metabolism.

Direct analysis of the intracellular content provides valuable insight into the cellular state including differentiation stage, metabolic state, and overall health.⁹ In the context of biomanufacturing, knowledge of the cell state can provide critical insight into the safety, efficacy, and potency of the final cell therapy product. Greater understanding of the intracellular biochemical environment can be leveraged to reduce developmental timelines in research settings by identifying CQAs and generating associated models at the systems biology level.¹⁰ These models can in turn be used to optimize production processes by identifying, tracking, and controlling critical process parameters (CPPs) and their resulting impact on critical quality attributes (CQAs) of cell therapies.¹¹

Existing intracellular analysis capabilities are largely constrained to downstream, targeted assays capable of detecting a limited number (and often only the most abundant) biochemicals. With over 23,000 unique biochemicals identified so far in the human metabolome, these methods are inadequate to fully characterize the cell state at a single time point, much less provide sufficient information for system level biology or in-process monitoring and control.¹² As such, significant interest has been shown in the development and deployment of existing offline analytical technologies for intracellular characterization to increase the breadth and depth of biochemical coverage. Given the sensitivity and specificity of mass spectrometry (MS) over a broad range of biochemical molecular weight, class, and concentration, MS is well positioned to provide detailed analysis of the intracellular state.^{9,13} Specifically, electrospray ionization mass spectrometry (ESI-MS) takes advantage of liquid phase ionization amenable for direct infusion analysis of complex mixtures. At this time, however, sample preparation requirements limit the capabilities of ESI-MS to offline applications and preclude its use for in-process monitoring.

Ideally, an MS-based workflow would provide broad biochemical coverage with minimal sample preparation from minimal sample sizes. Conventional intracellular mass spectrometry workflows, however, require tedious, manual, and time-consuming sample preparation.^{14,15} The limited throughput of such workflows restricts the data available for system level biology and CPP/CQA identification needed for the development of new cell-based therapies.¹⁶ With lengthy processing times, these workflows are also unable to capture the dynamic and heterogeneous nature of in vitro cell growth, especially internal metabolic processes with times scales on the order of minutes.¹⁷ The various preparatory steps (e.g., rinse, spin down, extraction, and concentration) are also performed in a batch manner and are not suitable for integration in a partially or fully automated production environment. In contrast, ambient ionization mass spectrometry approaches have demonstrated highly sensitive and specific biochemical detection directly from samples with minimal sample preparation.¹⁸⁻²² However, the initial extraction step of these techniques (whether solvent based or spatially resolved ionization) is a pre-treatment that targets only specific classes of biochemicals based on solubility or location in the sample. These techniques are also largely limited to applications with spatial access to immobilized cells or whole tissue samples and are not conducive to in-process monitoring of cell production systems such as stirred tank bioreactors.

Even if sample pre-treatment were optimized and reduced, the need for significant numbers of cells (ranging from hundreds of thousands to millions)¹⁴ to provide sufficient analytes for detection following sample preparation prohibits frequent, near continuous, in-process sampling directly from cell cultures. The need for such a large number of cells also prevents MS from being used as a quality control measure for donor qualification or product release of cell therapies given the irreplaceable value of each cell at both early and late time points of biomanufacturing. The sample volume needed for manual manipulation alone could result in significant perturbations of smaller production systems, especially for autologous cells, if frequent monitoring were desired. The pursuit of reduced sample size requirements has led to the emergence of single cell mass spectrometry. This field has generated important advances regarding processes internal to the cell by detection of both metabolites and proteins from individual cells. The rapidly growing body of literature on single cell MS highlights its capabilities for biomarker identification and biochemical pathway modelling in research settings.²³⁻²⁶ These techniques, however, rely heavily on elaborate cell micro-manipulation to ensure only the intracellular environment is sampled, which limits the utility of these techniques for in-line continuous monitoring of the cell population state in a biomanufacturing workflow.

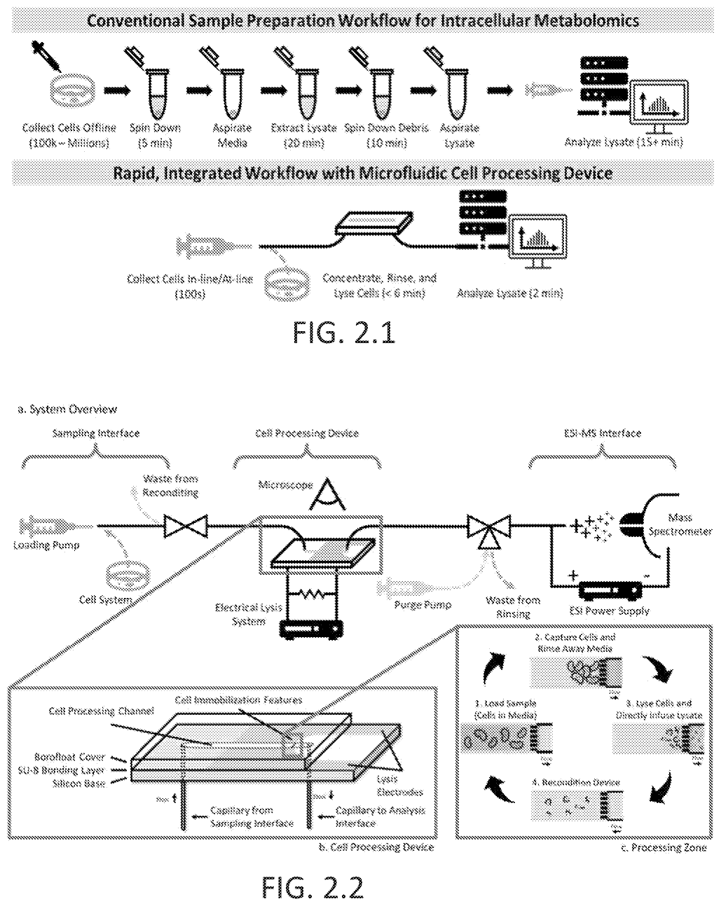

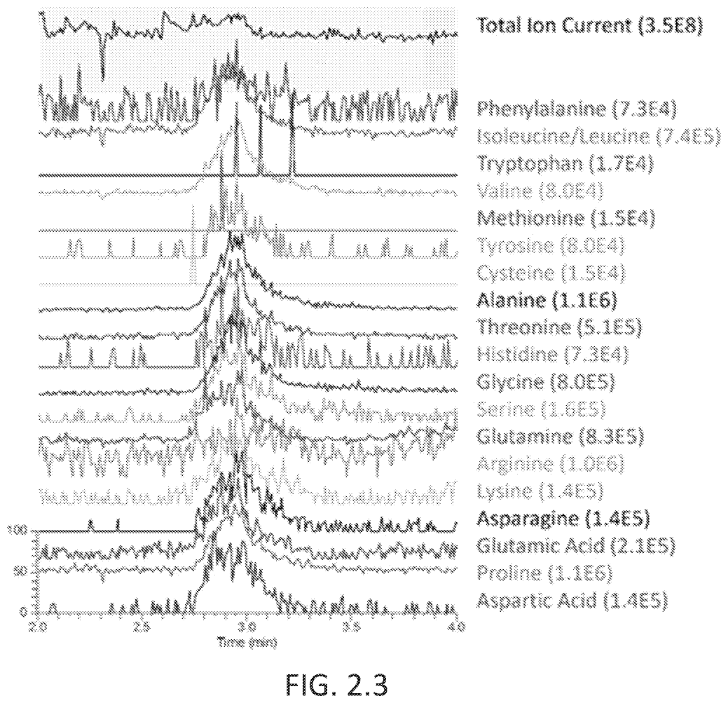

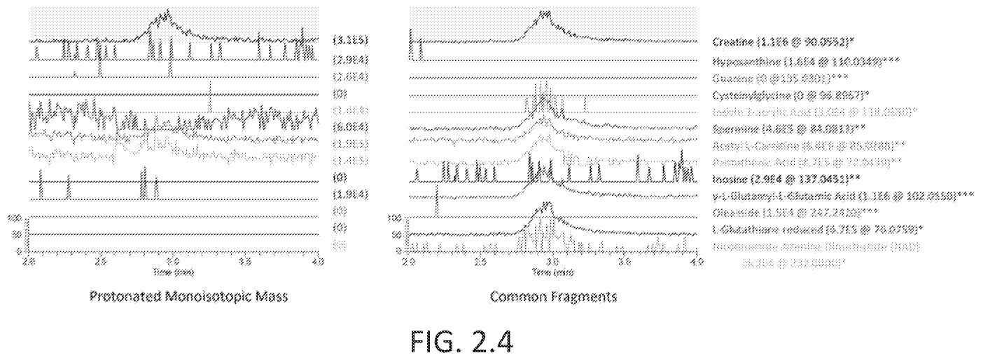

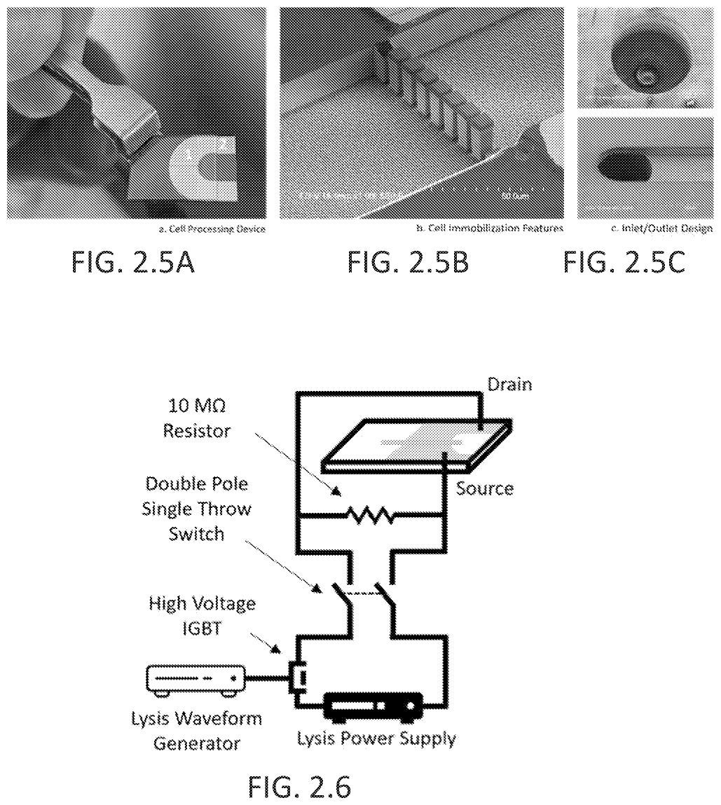

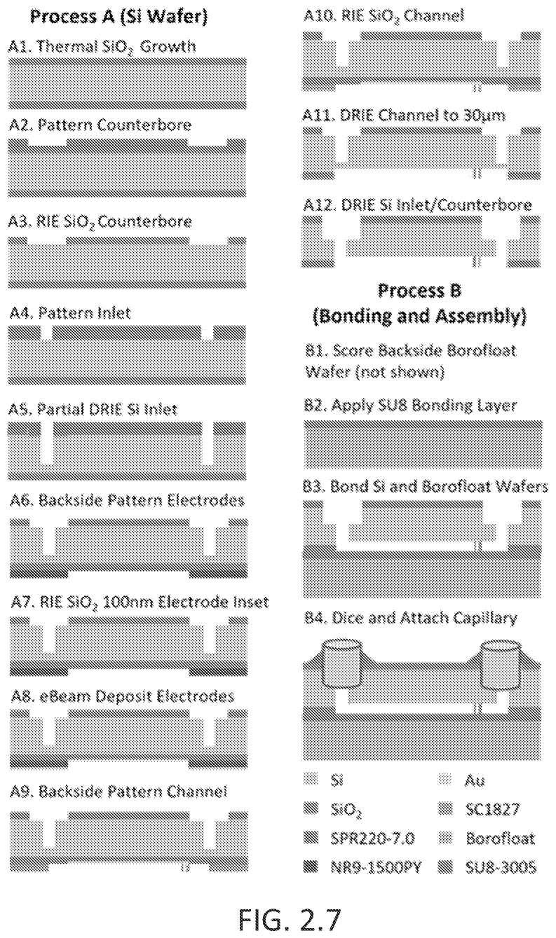

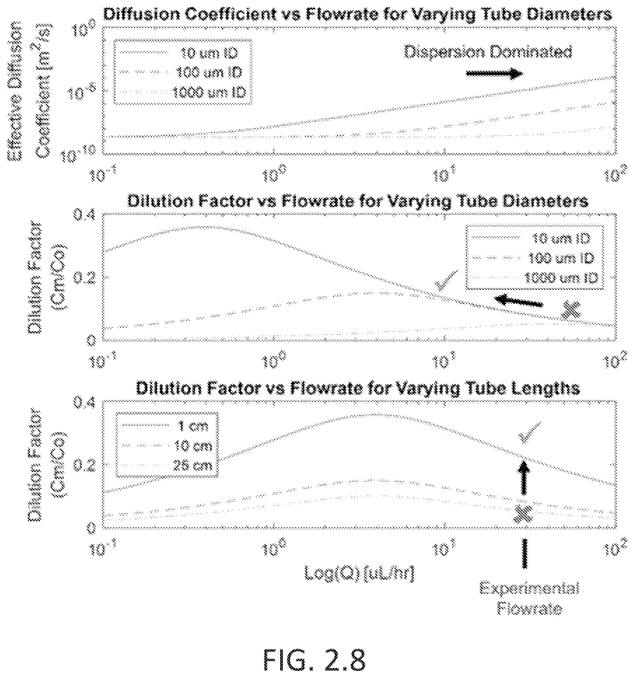

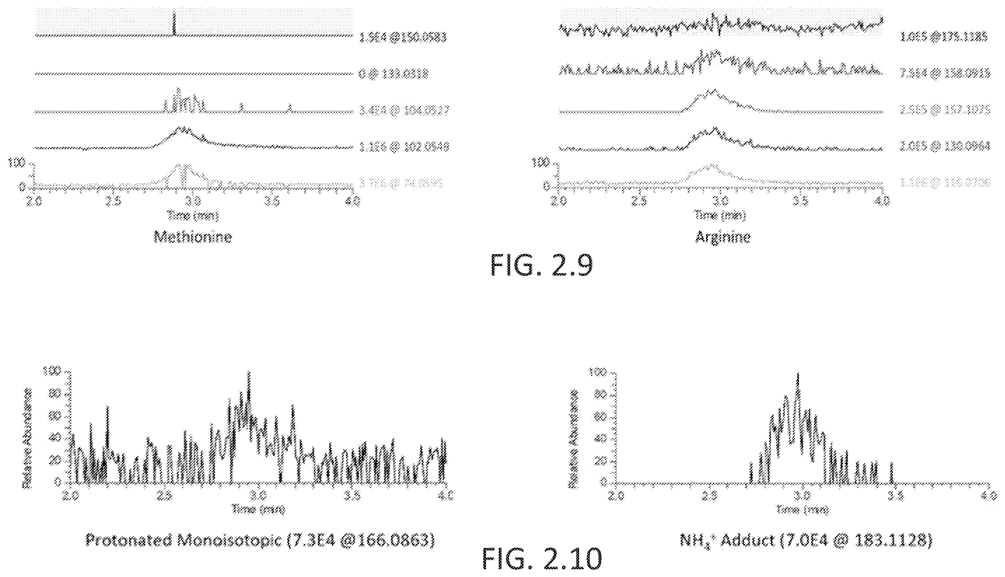

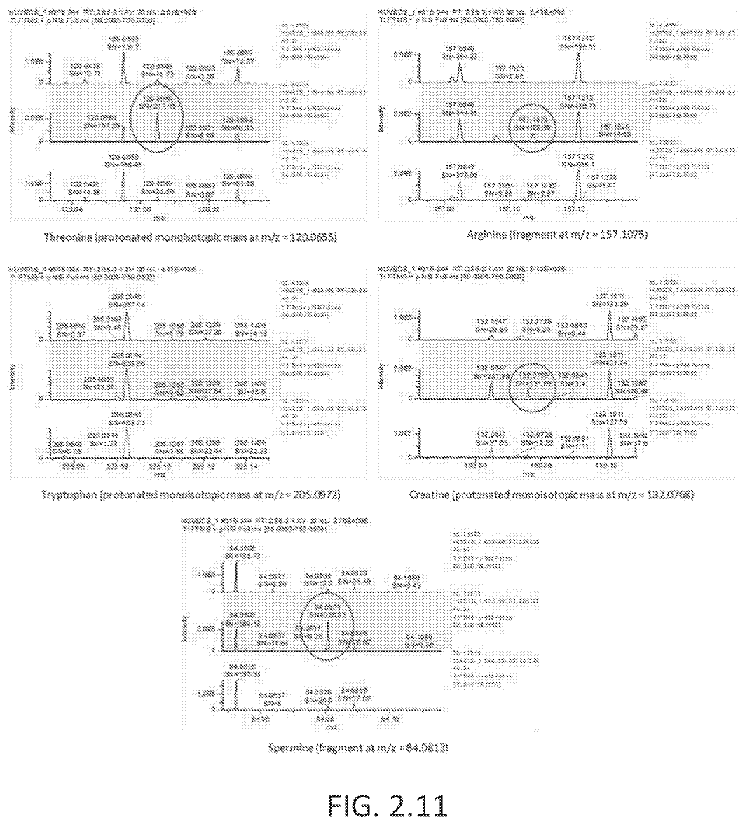

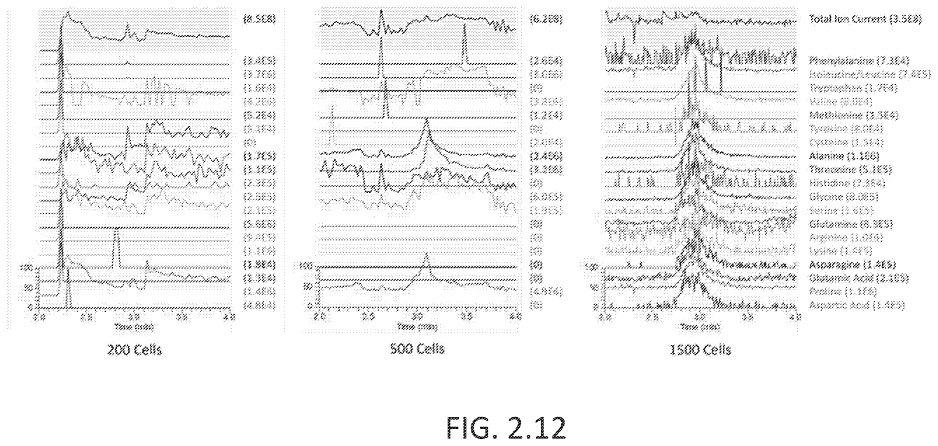

Though significant efforts have been made to increase throughput, minimize analysis times, reduce sample size, and eliminate manual handling through automation of the various aspects of conventional intracellular mass spectrometry (including in-line/at-line sample uptake, sample preparation, and sample introduction), fully integrated and rapid sample-to-analysis MS workflows have remained elusive.^{13,27-29} The mismatch between the highly variable, complex, and dynamic cell systems that need to be analyzed and the monitoring capabilities of mass spectrometry must be overcome to enable rapid intracellular analysis directly from culture. In this work, we present a new sample-to-analysis platform to effectively bridge the gap between cell system evolution and real-time MS analysis by taking small samples of cells directly from culture and performing a minimum number of processing steps for near continuous monitoring as shown in FIG. 2.1. Rather than focusing on the comprehensive analysis (as is the goal of conventional workflows) or cell-by-cell targeted investigation (as is the goal of single cell workflows), this new process analytical technology enables dynamic characterization of the cell state directly from the growth environment with untargeted detection of a sufficiently broad range of relevant biomarkers, including intracellular metabolites.

Central to the platform is a microfluidic cell processing device capable of preparing ultra-small cell samples (on the order of hundreds of cells compared to hundreds of thousands needed for conventional workflows) for direct infusion ESI-MS. The device incorporates the critical aspects of conventional MS intracellular workflows (e.g., isolation, rinsing, and extraction) in a flow through format for rapid (less than 10 minute) analysis with no manual handling following sample uptake. The microfluidic design allows for minimum dilution of the sample prior to analysis; this is critical for achieving high sensitivity while requiring minimal cell samples. The device is coupled to a microcapillary sampling probe for direct-from-culture cell uptake (upstream) and an in-line nanoESI emitter for direct infusion to MS (downstream). By reducing the delay from “sampling to spectra”, the platform significantly increases the temporal resolution of bioprocess monitoring. The system is also designed such that it can be regenerated following each analysis cycle to provide near continuous monitoring in a single, integrated, and reusable format. These capabilities, alone or in combination with other approaches (e.g., secretome and transcriptome analyses), enable enhanced control of cell processes for both basic research applications as well as clinical production. The following study demonstrates the utility of this platform for intracellular characterization of cell therapies with focus on: 1) overview of the platform, including key design aspects; 2) demonstration of the utility of the workflow for detecting intracellular metabolites directly from culture; 3) system design and operating conditions, including microfabrication details, that enable dynamic monitoring using small numbers of cells.

### Integrated Microfluidic Workflow

As shown in FIG. 2.2A-C, the integrated workflow starts with cell uptake via the sampling interface followed by sample conditioning in the cell processing device before direct, in-line ESI-MS analysis. During sample uptake, a syringe pump withdraws the desired volume at flowrates on the order of 50 nL/s from a cell culture system (e.g., well plates, culture dishes or flasks, stirred flask bioreactors) into the sample capillary. Extracting several hundred cells allows for sub-microliter volumes to be removed from the culture in a matter of seconds given typical cell culture concentrations. Removal of such small sample volumes has negligible impact on the total cell count or viability of the cell culture system, a critical requirement for frequent, in-process monitoring.

Following uptake, the cells are infused into the microfabricated cell processing device (FIG. 2.2B). Within the device, cells are immobilized and concentrated in the cell lysis region using cell capture features (FIG. 2.2C). Following capture, the concentrated cells are rinsed by continuously flowing 150 mM ammonium acetate. This rinse step removes the constituents of the cell culture media to ensure the analysis is solely representative of the intracellular content of cells, as well as eliminating ESI-MS interferants (e.g., salts) present at high concentrations in the cell media. Assuming complete dissolution, a 150 mM ammonium acetate solution translates to a 300 mOsm solution, approximating typical physiological osmolarity. Use of an iso-osmotic solution prevents uncontrolled osmotic lysis during rinsing which would result in time dependent release of the intracellular contents and subsequent dilution.³⁰ Furthermore, as a volatile additive, ammonium acetate reduces ion suppression during ESI-MS analysis compared to non-volatile salts present in the media and used in other isotonic buffers (e.g., PlasmaLyte).³¹

During rinsing, the flow downstream of the cell processing device is diverted to waste to prevent carryover effects and reduce clogging in the ESI emitter. Upon completion of a 3× volume rinse of the entire system, the flow is directed to the ESI emitter for direct infusion to the MS (approximately 4 minutes post sample collection). The intracellular contents are extracted via electrical pulses applied across the lysis electrodes (FIG. 2.2B), resulting in irreversible poration of the cell membrane approximately 6 minutes post sample collection.

Electrical lysis provides near-instantaneous release of the cellular contents, regardless of cell type, and is chosen in favor of chemically induced lysis techniques that could interfere with the detection of intracellular biochemicals.³²⁻³⁴ The lysis step is initiated only after a stable ESI flow is established to ensure consistency of ESI-MS analyses (4-6 minutes post sample collection). Upon completion of the analysis of an infused sample, the system is regenerated by flowing a reconditioning buffer in the reverse direction to purge the system of cell debris and residual species (FIG. 2.2C). This returns the system to its initial state, ready for subsequent analyses such that a single device can be used repeatedly.

Detection of Metabolites from Cell Culture

The analytical workflow was applied to detect intracellular metabolites from a sample of approximately 1500 human umbilical vein endothelial cells (HUVECs). FIG. 2.3 shows the resulting MS signal intensities for mass-to-charge values (m/z) corresponding to the protonated monoisotopic masses of the 20 amino acids found in the genetic code. As the building blocks of proteins and regulators of metabolism, amino acids represent an essential class of metabolites regardless of cell type.³⁵ Significant deviations from baseline physiological ranges (both excess and deficient) of particular acids can serve as disease markers for illnesses ranging from metabolic disorders³⁶ to neurodegenerative diseases.³⁷ Many amino acids have also been identified as CQAs for the development and characterization of new cell therapies, being up or down regulated in response to culture conditions³⁸ and serving as critical targets in disease modelling studies.³⁹ Amino acids also represent a diverse subset of metabolites ranging from hydrophobic to hydrophilic, having both charged and neutral species, and being present across several orders of magnitude of concentration within the cell, thus representing a comprehensive and clinically relevant testbed.

The top trace of FIG. 2.3 represents the total ion current (TIC) for each MS scan; it displays minimal variation in the time period immediately following lysis, indicating stable ESI. Below the TIC, the amino acid traces are shown. The nearly constant TIC allows for interpretation of increases of signal intensity at specific m/z values to indicate the presence of a given amino acid in the cell lysate. Fourteen out of nineteen of the protonated monoisotopic mass traces displayed distinct signal intensity increases (as isomers, leucine and isoleucine cannot be distinguished without additional separation/analysis schemes and are thus represented by a single trace). The increases align with the anticipated elution characteristics (time delay and duration of the peak) and thus provide strong evidence of successful detection. Five amino acids (phenylalanine, tryptophan, methionine, cysteine, and arginine) showed either no or inconclusive increases in signal intensity. Given the mechanisms of ESI-MS, it is possible an analyte is present but not detectable at the protonated monoisotopic mass. In such cases, tracing possible adducts (i.e., amino acid plus Na⁺, K⁺, and NH₄⁺) and potential fragmentation patterns provides additional means of detection. Open-source databases (MassBank of North America, MassBank Europe, and MZMine) were queried for common fragments identified using ESI-MS and compared against the sample spectra of the five undetected amino acids. Both methionine and arginine displayed distinct increases associated with the identified fragments (FIG. 2.9). While no fragments of phenylalanine were identified, the NH₄⁺ adduct mass displayed a distinct signal increase (FIG. 2.10). This is consistent with the presence of NH₄⁺ ions in solution following disassociation of ammonium acetate which is present in the rinsing buffer.

Even after tracking the protonated monoisotopic, fragment, and adduct masses, the absence of a distinct signal increase does not necessarily indicate the absence of an analyte. The ESI-MS analysis of multi-analyte solutions is dependent on a complex interplay of ionization potential, charge scavenging, and limit of detection for a given analyte. These complications are further exaggerated in complex solutions such as the cell lysate which is expected to contain thousands of electro-chemically distinct analytes distributed across several orders of magnitude in concentration. For example, cysteine and tryptophan are among the least abundant amino acids, and therefore likely fall below the limit of detection even though they are expected to be present within the cell.⁴⁰

To further demonstrate the analytical capability of the sample-to-analysis platform, a more diverse segment of the metabolome clinically relevant specifically to HUVECs has been analyzed as shown in FIG. 2.4. Jayaraman et al. identified intracellular metabolites significantly up or down-regulated when HUVECs were co-cultured with either highly metastatic or highly invasive prostate cancer cells.³⁹ These metabolites represent potential biomarkers and may provide targets for inhibiting cancer growth through metabolic control of endothelial cells in the tumor microenvironment. FIG. 2.4 depicts the thirteen non-amino acid metabolite markers identified by Jayaraman et al. using positive mode ESI-MS with upstream high-performance liquid chromatography (HPLC); the five amino acids (L-glutamic acid, L-arginine, L-tryptophan, L-tyrosine, and methionine) identified in the study are included in the results of FIG. 2.3. Of the protonated monoisotopic traces shown on the left of FIG. 2.4 (sorted by descending m/z), only creatine showed a distinct elution band. Extending the analysis to include common fragments reported in open-source databases, seven additional analytes displayed distinct signal increases corresponding to the lysate elution band as seen on the right of FIG. 2.4. In total, twelve of the eighteen metabolites (four amino acids and eight non-amino acid metabolites) identified by Jayaraman et al. were detected by our system. The remaining six undetected metabolites (hypoxanthine, guanine, cysteinylglycine, inosine, oleamide, and nicotinamide adenine dinucleotide) are likely missed due to charge scavenging, abundance below limit of detection, and/or ion suppression during ESI. Representative spectra of both detected and undetected metabolites are included in FIG. 2.11.

### Effect of Sampled Number of Cells on Metabolite Detection

To characterize the sensitivity of the system to the number of cells up-taken, samples containing approximately 200, 500, and 1500 cells were analyzed. For the case of 200 cells, none of the traced analytes were detected in the anticipated lysate band. For the case of 500 cells, only three of the nineteen amino acids showed distinct signal increases at the protonated monoisotopic mass as shown in FIG. 2.12. For 1500 cells, 14 out of 19 amino acids were detected at the protonated monoisotopic mass as discussed in the previous sections.

The sensitivity to cell number is due both to system limitations and the broad dynamic range of intracellular metabolite concentrations. The number and concentration of intracellular molecules per cell varies for each species ranging from vanishingly small up to 10¹¹ (mM range) molecules per cell.²³ The limit of detection also varies between analytes as determined by ionization potential and susceptibility to in-source modifications (i.e., adduct formation or fragmentation). Regarding system operation, the dilution factor following lysis is independent of cell number as the cells form a packed bed during the immobilization step. The dilution factor following dispersion is, however, dependent on the initial width of the lysate band, and thus the cell number. For the system described herein, the final dilution factor is approximately 0.4% for analysis of 100 cells. For a metabolite at an intracellular concentration of 1 μM, the average concentration in the dispersed lysate band at the emitter would be a mere 4 nM. Using a greater number of cells significantly reduces the final dilution, increasing the final concentration by nearly an order of magnitude for the case of 1000 cells (dispersion calculation details can be found in Supplementary Information). As such, the combination of both system and species dependent sensitivity yields non-uniform behavior in reaching the limit of detection across all analytes as a function of the number of cells used in the analysis.

Even with this unavoidable dependence on cell number, the developed sample-to-analysis platform detected seventeen out of nineteen amino acids and a majority (twelve out of eighteen) of HUVEC specific biomarkers from a very small sample of just 1500 cells. This is a significant analytical result, given that it was achieved using a small number of cells (vs hundreds of thousands or more) in a matter of minutes (versus hours) compared to conventional HPLC ESI-MS workflows. Collectively, these results demonstrate the capability for the developed microfluidic platform to operate in a quasi-continuous flow format for rapid assessment of the intracellular metabolome with broad biochemical coverage.

### Fabrication, Characterization, and Operation

### Microfabrication of Cell Processing Device

The cell processing device is manufactured using advanced microfabrication techniques, enabling integration of numerous features in a single device with opportunities for scaled production via batch processing. The details of the process are given in Supplementary Information with key elements of the design and processing sequence summarized here. Thirty-two devices, each with 10 mm×15 mm footprint, are fabricated on a 4″ diameter, 500 μm thick silicon wafer. The bulk of processing is centered around creation of microfluidic channels 5.075 mm long, 100 μm wide, and 30 μm deep. A series of parallel pillars spanning the channel width, each 5 μm wide with 3 μm spacing, serves as the cell immobilization feature (FIGS. 2.5A-C). This design effectively captures and concentrates the cells at the restriction point while allowing the media and rinsing buffer to continuously flow, thus avoiding clogging and high-pressure conditions that can lead to device delamination. Lysis electrodes are positioned along the channels and extend 3.975 mm upstream of the cell immobilization features. Gold was used as the electrode material as it will not corrode when exposed to the sample solution (the electrodes are in direct contact with fluid in the channel) and has favorable electromigration properties to withstand the repetitive application of high voltages applied across the thin layer. Inlet and outlet holes, 100 μm in ID, are etched through the silicon at the extents of the channel. Concentric to the through holes are 360 μm diameter counterbores which are etched approximately 250 μm deep. This design enables robust incorporation of 360 μm OD capillary inlet and outlet tubing for low dead volume fluidic connections.⁴¹ The total device volume is 17 nL.

The channels are capped with a Borofloat 33 (Schott, Rye Brook, NY) cover. Borofloat is transparent and allows flow visualization within the device while having a coefficient of thermal expansion on the same order of magnitude as silicon to ensure thermomechanical compatibility during the bonding step. The Borofloat fully covers the fluidic channel while leaving a portion of the electrodes exposed at the edges of the device to serve as electrical pads for application of the lysis pulses. The Borofloat is bonded to the silicon wafer using an SU8-3005 adhesive layer (MicroChem, Westborough, MA). SU8 was chosen as it provides a water-insoluble, chemically inert bonding layer. Numerous studies have shown SU8 compatibility with MS, demonstrating it is resistant to common MS solvents, will not leach into the analysis fluid (resulting in polymer peaks), nor scavenge biochemicals from the sample by absorption⁴² in contrast to other microfluidic device materials such as polydimethylsiloxane (PDMS).⁴³⁻⁴⁵ Several devices were exposed to the electrical lysis pulse sequence with only buffer in the device to investigate the potential breakdown of SU8 in the presence of high electric fields. The resulting spectra showed no contaminants were released during the tests. This conclusion is in agreement with the electric breakdown field of SU8 which is reported as approximately 10⁸ V/m, three orders of magnitude above the 6 kV/cm lysis pulse.⁴²

The devices proved to be robust and reusable for multiple sequential measurements with no detectable carry-over between the runs. However, the risk of carryover in analysis of complex samples with such large ranges of biochemical concentrations is notable and could impact the reliability of the analytical output. Batch fabrication allows for dramatic reduction in the cost per device such that even single use becomes practical given the reduced burden of cell number, analytical effort, and analysis time. Following dicing of individual devices and attachment of inlet/outlet capillaries, the finished cell processing devices are held in a custom machined plexiglass fixture to facilitate orientation in front of the MS, ease connection of the lysis circuit to the electrodes, and allow real-time visualization of the channel via a digital microscope.

### Fluidic System

With reference to FIG. 2.2, the various fluidic components connecting the sampling and analysis interfaces to the cell processing device were designed to balance sample transit time, dead volume, pressure drop, and diffusion/dispersion effects. Detailed assessment of the trade-offs is given in Supplementary Information with the specific configuration detailed here. A LabSmith CapTite MV201-C360 3-port selector valve (LabSmith, Livermore, CA) with 130 nL swept volume connects the cell processing device to the flow conduit for sample regeneration and to the ESI emitter via 360 μm OD capillary. Other non-valved connections (e.g., between syringe and fluidic system) are via Valco ZU1XC zero volume unions (VICI Valco Instruments, Houston, TX). A 360 μm OD, 75 μm ID fused silica capillary is used upstream of the device to accommodate cell loading without clogging. The length of sampling capillary is such that the entire sampled volume is contained within; this allows for the syringe pump to be used in the withdraw/infuse manner without introducing cells into the syringe itself. Downstream of the device, 50 μm ID fused silica capillary is used to reduce transit time of the lysate volume while minimizing clogging and pressure drop. The length of capillary between the cell processing device and ESI emitter, as well as the emitter itself, are minimized to further reduce transit time, pressure drop, and dispersion effects.

### Electrical Lysis Configuration and Operation

Lysis pulses are applied between the electrodes lining the cell processing channel as seen in FIG. 2.5A. Prior to the lysis sequence, the leads of the lysis power supply are held at the same potential until a high voltage insulated gate bipolar transistor (IGBT) gate is closed according to the chosen pulse parameters (voltage, duration, and frequency) (FIG. 2.6). Closing the gate completes the circuit internal to the lysis system, effectively draining the current across the high resistance network formed by the resistor and microfluidic channel. With electric leads on each side of the network, one electrode becomes the “source” and the other, the “drain”. This results in an electric potential difference between the electrodes sufficient for lysis. Once the gate is re-opened, there is no current flow and thus no voltage drop across the resistance network, allowing the “drain” to again float to an equal potential as the “source”. The lysis voltage is applied via a Stanford Research Systems PS350 High Voltage Power Supply (Stanford Research Systems, Sunnyvale, CA). The pulse duration, shape, and frequency are controlled by an Agilent 33250A waveform generator (Keysight Technologies, Santa Rosa, CA); a 5 Vpp, +2.5 V DC offset output signal is supplied to fully open and close the IGBT gate. An IXYS IXYL60N450 IGBT (Littelfuse, Chicago, IL) enables high voltage, high power control with nanosecond switching times (MHz switching frequency). When not in operation, the entire lysis circuit is electrically isolated from the ESI electrical circuit by a switch to prevent uncontrolled electrolysis in the system, ensuring stability of the ESI-MS signal.

The lysis efficiency depends on an optimal combination of pulse amplitude, duration, and number for a given electrode configuration.⁴⁶ The applied voltage was set at 60 V, corresponding to an electric field across the 100 μm channel of 6 kV/cm. This value is within the electric field strength required for irreversible electroporation of mammalian cells (typically reported as >1 kV/cm or >1 V in terms of transmembrane potential).^{46,47} The pulse duration was then incrementally increased from microseconds to milliseconds and the extent of electrolysis in the channel was monitored. The pulse sequence of 1000, 5 ms square waves applied at 100 Hz was selected such that observable electrolysis occurred in the channel but did not result in bubbles that spanned the channel. Such limited electrolysis showed no appreciable impact on the ESI-MS signal but provides a visual cue that electrical lysis is performed. The combination of voltage and pulse sequence is in agreement with observations that longer duration pulses, even when applied at lower voltages, result in higher lysis efficiency compared to stronger but shorter duration pulses.⁴⁷ The efficiency further increases with a greater number of pulses, but must be balanced with the extent of electrolysis allowable to maintain ESI stability.

### Summary of Experimental Demonstrations

Mass spectrometry holds unparalleled potential for ultra-sensitive and specific intracellular biochemical analysis. At present, however, no MS based analytical technology is available that allows for continuous, temporally resolved monitoring of the intracellular metabolome. Further, conventional MS metabolomics is limited to working with relatively large samples containing extracts from hundreds of thousands of cells. This work presents a microfluidic platform and associated workflow to overcome these limitations. This integrated sample-to-analysis platform was applied to the intracellular analysis of 1500 HUVEC cells in native media sampled directly from a cell suspension system. The analysis enables detection of nearly all proteogenic amino acids as well as a majority of key metabolites identified as HUVEC specific biomarkers. Important to workflow validation, the developed platform has demonstrated the capability to detect clinically relevant intracellular biomarkers which have been previously identified using conventional HPLC ESI-MS. In addition to its demonstrated analytical power, the platform replaces the numerous manual handling steps with paths toward complete automation, works with ultra-small cell samples, is capable of self-regeneration for long-term, continuous operation, and is suitable for integration into cell growth bioreactors for direct-from-culture analysis. Continuous biochemical readout of the intracellular environment in real-time, as demonstrated in this work, is a critical milestone to enable fully automated quality monitoring with integrated feedback control in cell-based therapy manufacturing.

### Experimental Methods

### HUVEC Culture and Harvesting

Human umbilical vein endothelial cells (Lonza, Basel, Switzerland) were cultured in EGM-2MV (Lonza) on 150 cm² tissue culture flasks coated with 0.1% gelatin (Sigma-Aldrich, St. Louis, MO), in a 37° C. and 5% CO₂ incubator. Cells were harvested at passage 6 by rinsing with 1 mL/25 cm² phosphate-buffered saline (Corning, Corning, NY), incubating with 1 mL/25 cm² TrypLE (Thermo Fisher Scientific, Waltham, MA), and neutralizing with 1 mL/25 cm² 10% fetal bovine serum (Cytiva, Marlborough, MA) in PBS. Cells were then pelleted at 300 g for 5 minutes at 4° C., resuspended in 1 mL/75 cm² EGM-2MV, and counted using a Countess Automated Cell Counter (Thermo Fisher Scientific) using AOPI live cell discrimination (Nexcelom Bioscience, Lawrence, MA).

### ESI-MS Analysis and Data Processing

Prior to analysis, the system was primed with the buffer flow to eliminate any bubbles that might disrupt ESI. Cells were sampled directly from a suspension in native media at an uptake flowrate of 150 μL/hr. The sample was loaded into the cell processing device and immediately rinsed by continuously flowing rinsing buffer for 4 minutes. The rinsing duration corresponds to a 3× rinse of the entire system volume to ensure all media components are purged from the system. During the rinse, the downstream flow was diverted from the ESI emitter and directed to waste. Following the rinse, the flow was directed to the ESI emitter via the switching valve and the ESI voltage of ˜4 kV was applied until stable ESI was established. Once stable ESI was observed (variation in total ion current less than 20%), the ESI voltage was turned off, the lysis circuit activated, lysis pulses applied approximately 2 min post rinse, and ESI immediately reinitiated for analysis. Protonation of analytes is promoted by addition of 0.1% acetic acid by volume to the buffer solution.

Data was acquired using a Thermo Scientific Q Exactive Plus Quadrupole-Orbitrap mass spectrometer (Thermo Fisher Scientific). The MS was operated in full scan positive mode with a mass range of 50-750 m/z and resolving power of 140,000 FWHM at 200 m/z. The automatic gain control (AGC) target was set to 1E6 with a maximum injection time of 500 ms; the S-Lens RF level was set to 40 to reduce fragmentation and ensure sensitivity at lower m/z values. Fused silica ESI emitters were fabricated in house from 360 μm OD, 100 μm ID capillary. The capillary was pulled to a fine point tip using a Sutter P-2000 Laser-Based Micropipette Puller System (Sutter Instrument, Novato, CA). The internal diameter of the emitter was enlarged by trimming the tip using an Optec Femtosecond laser (Optec Laser Systems, San Diego, CA); the laser allows for precise control of the final tip dimensions with a target ID of 15 μm to prevent clogging of the emitter while still enabling stable nanoESI. The emitter orientation in front of the MS and ESI voltage were adjusted until the AGC target was reached for each scan and variability in the total ion current was less than 20%. Data analysis was performed using Thermo Scientific FreeStyle software. Targeted analyses were performed using mass traces with 10 ppm mass tolerance for each anticipated m/z value. The protonated monoisotopic mass of each target analyte was initially queried with secondary traces according to m/z values of fragments reported in MassBank of North America (MoNA), MassBank Europe, or MZMine databases.

### REFERENCES FOR EXAMPLE 1

1. Pittenger, M. F.; Discher, D. E.; Péault, B. M.; Phinney, D. G.; Hare, J. M.; Caplan, A. I., Mesenchymal stem cell perspective: cell biology to clinical progress. npj Regenerative Medicine 2019, 4 (1), 22.
2. Sermer, D.; Brentjens, R., CAR T-cell therapy: Full speed ahead. Hematological Oncology 2019, 37 (Si), 95-100.
3. Vismara, I.; Papa, S.; Rossi, F.; Forloni, G.; Veglianese, P., Current Options for Cell Therapy in Spinal Cord Injury. Trends in Molecular Medicine 2017, 23 (9), 831-849.
4. Aijaz, A.; Li, M.; Smith, D.; Khong, D.; LeBlon, C.; Fenton, O. S.; Olabisi, R. M.; Libutti, S.; Tischfield, J.; Maus, M. V.; Deans, R.; Barcia, R. N.; Anderson, D. G.; Ritz, J.; Preti, R.; Parekkadan, B., Biomanufacturing for clinically advanced cell therapies. Nature Biomedical Engineering 2018, 2 (6), 362-376.
5. Roh, K.-H.; Nerem, R. M.; Roy, K., Biomanufacturing of Therapeutic Cells: State of the Art, Current Challenges, and Future Perspectives. Annual Review of Chemical and Biomolecular Engineering 2016, 7 (1), 455-478.
6. Biechele, P.; Busse, C.; Solle, D.; Scheper, T.; Reardon, K., Sensor systems for bioprocess monitoring. Engineering in Life Sciences 2015, 15 (5), 469-488.
7. Abu-Absi, N. R.; Kenty, B. M.; Cuellar, M. E.; Borys, M. C.; Sakhamuri, S.; Strachan, D. J.; Hausladen, M. C.; Li, Z. J., Real time monitoring of multiple parameters in mammalian cell culture bioreactors using an in-line Raman spectroscopy probe. Biotechnology and Bioengineering 2011, 108 (5), 1215-1221.
8. Landgrebe, D.; Haake, C.; Höpfner, T.; Beutel, S.; Hitzmann, B.; Scheper, T.; Rhiel, M.; Reardon, K. F., On-line infrared spectroscopy for bioprocess monitoring. Applied Microbiology and Biotechnology 2010, 88 (1), 11-22.
9. Dettmer, K.; Aronov, P. A.; Hammock, B. D., Mass spectrometry-based metabolomics. Mass spectrometry reviews 2007, 26 (1), 51-78.
10. May, M., Big data, big picture: Metabolomics meets systems biology. Science Magazine 2017, pp 646-648.
11. Consortium, N. C. M. Achieving Large-Scale, Cost-Effective, Reproducible Manufacturing of High-Quality Cells: A Technology Road Map to 2025; Office of Science, Technology, and Policy: 2016.
12. Wishart, D. S.; Feunang, Y. D.; Marcu, A.; Guo, A. C.; Liang, K.; Vázquez-Fresno, R.; Sajed, T.; Johnson, D.; Li, C.; Karu, N.; Sayeeda, Z.; Lo, E.; Assempour, N.; Berjanskii, M.; Singhal, S.; Arndt, D.; Liang, Y.; Badran, H.; Grant, J.; Serra-Cayuela, A.; Liu, Y.; Mandal, R.; Neveu, V.; Pon, A.; Knox, C.; Wilson, M.; Manach, C.; Scalbert, A., HMDB 4.0: the human metabolome database for 2018. Nucleic Acids Res 2018, 46 (D1), D608-D617.
13. Lombard-Banek, C.; Schiel, J. E., Mass Spectrometry Advances and Perspectives for the Characterization of Emerging Adoptive Cell Therapies. Molecules 2020, 25 (6), 1396.
14. Yuan, M.; Breitkopf, S. B.; Yang, X.; Asara, J. M., A positive/negative ion-switching, targeted mass spectrometry-based metabolomics platform for bodily fluids, cells, and fresh and fixed tissue. Nat Protoc 2012, 7 (5), 872-81.
15. Courant, F.; Antignac, J.-P.; Dervilly-Pinel, G.; Le Bizec, B., Basics of mass spectrometry based metabolomics. Proteomics 2014, 14 (21-22), 2369-2388.
16. Majumder, E. L. W.; Billings, E. M.; Benton, H. P.; Martin, R. L.; Palermo, A.; Guijas, C.; Rinschen, M. M.; Domingo-Almenara, X.; Montenegro-Burke, J. R.; Tagtow, B. A.; Plumb, R. S.; Siuzdak, G., Cognitive analysis of metabolomics data for systems biology. Nature Protocols 2021, 16 (3), 1376-1418.
17. Shamir, M.; Bar-On, Y.; Phillips, R.; Milo, R., SnapShot: Timescales in Cell Biology. Cell 2016, 164 (6), 1302-1302.e1.
18. Rappez, L.; Stadler, M.; Triana, S.; Gathungu, R. M.; Ovchinnikova, K.; Phapale, P.; Heikenwalder, M.; Alexandrov, T., SpaceM reveals metabolic states of single cells. Nature Methods 2021, 18 (7), 799-805.
19. Nemes, P.; Vertes, A., Laser Ablation Electrospray Ionization for Atmospheric Pressure, in Vivo, and Imaging Mass Spectrometry. Analytical Chemistry 2007, 79 (21), 8098-8106.
20. Takáts, Z.; Wiseman, J. M.; Gologan, B.; Cooks, R. G., Mass Spectrometry Sampling Under Ambient Conditions with Desorption Electrospray Ionization. Science 2004, 306 (5695), 471-473.
21. Cohen, L. H.; Gusev, A. I., Small molecule analysis by MALDI mass spectrometry. Analytical and Bioanalytical Chemistry 2002, 373 (7), 571-586.
22. Feider, C. L.; Krieger, A.; DeHoog, R. J.; Eberlin, L. S., Ambient Ionization Mass Spectrometry: Recent Developments and Applications. Analytical Chemistry 2019, 91 (7), 4266-4290.
23. Zhang, L.; Vertes, A., Single-Cell Mass Spectrometry Approaches to Explore Cellular Heterogeneity. Angew Chem Int Ed Engl 2018, 57 (17), 4466-4477.
24. Duncan, K. D.; Fyrestam, J.; Lanekoff, I., Advances in mass spectrometry based single-cell metabolomics. Analyst 2019, 144 (3), 782-793.
25. Hofstadler, S. A.; Severs, J. C.; Smith, R. D.; Swanek, F. D.; Ewing, A. G., Analysis of single cells with capillary electrophoresis electrospray ionization Fourier transform ion cyclotron resonance mass spectrometry. Rapid Commun Mass Spectrom 1996, 10 (8), 919-22.
26. Kawai, T.; Ota, N.; Okada, K.; Imasato, A.; Owa, Y.; Morita, M.; Tada, M.; Tanaka, Y., Ultrasensitive Single Cell Metabolomics by Capillary Electrophoresis-Mass Spectrometry with a Thin-Walled Tapered Emitter and Large-Volume Dual Sample Preconcentration. Analytical Chemistry 2019, 91 (16), 10564-10572.
27. Gao, J.; Yin, X.-F.; Fang, Z.-L., Integration of single cell injection, cell lysis, separation and detection of intracellular constituents on a microfluidic chip. Lab on a Chip 2004, 4 (1), 47-52.
28. Filla, L. A.; Sanders, K. L.; Filla, R. T.; Edwards, J. L., Automated sample preparation in a microfluidic culture device for cellular metabolomics. Analyst 2016, 141 (12), 3858-3865.
29. Li, X.; Zhao, S.; Hu, H.; Liu, Y. M., A microchip electrophoresis-mass spectrometric platform with double cell lysis nano-electrodes for automated single cell analysis. J Chromatogr A 2016, 1451, 156-163.
30. Berman, E. S. F.; Fortson, S. L.; Checchi, K. D.; Wu, L.; Felton, J. S.; Wu, K. J. J.; Kulp, K. S., Preparation of Single Cells for Imaging/Profiling Mass Spectrometry. Journal of the American Society for Mass Spectrometry 2008, 19 (8), 1230-1236.
31. Sterling, H. J.; Batchelor, J. D.; Wemmer, D. E.; Williams, E. R., Effects of Buffer Loading for Electrospray Ionization Mass Spectrometry of a Noncovalent Protein Complex that Requires High Concentrations of Essential Salts. Journal of the American Society for Mass Spectrometry 2010, 21 (6), 1045-1049.
32. Shehadul Islam, M.; Aryasomayajula, A.; Selvaganapathy, P. R., A Review on Macroscale and Microscale Cell Lysis Methods. Micromachines 2017, 8 (3), 83.
33. Geng, T.; Lu, C., Microfluidic electroporation for cellular analysis and delivery. Lab on a Chip 2013, 13 (19), 3803-3821.
34. Grigorov, É.; Kirov, B.; Marinov, M.; Galabov, V., Review of Microfluidic Methods for Cellular Lysis. Micromachines 2021, 12.
35. Krumpochova, P.; Bruyneel, B.; Molenaar, D.; Koukou, A.; Wuhrer, M.; Niessen, W. M. A.; Giera, M., Amino acid analysis using chromatography-mass spectrometry: An inter platform comparison study. Journal of Pharmaceutical and Biomedical Analysis 2015, 114, 398-407.
36. Aliu, E.; Kanungo, S.; Arnold, G. L., Amino acid disorders. Ann Transl Med 2018, 6 (24), 471-471.
37. Socha, E.; Koba, M.; Kośliński, P., Amino acid profiling as a method of discovering biomarkers for diagnosis of neurodegenerative diseases. Amino Acids 2019, 51 (3), 367-371.
38. Doron, G.; Klontzas, M. E.; Mantalaris, A.; Guldberg, R. E.; Temenoff, J. S., Multiomics characterization of mesenchymal stromal cells cultured in monolayer and as aggregates. Biotechnology and Bioengineering 2020, 117 (6), 1761-1778.
39. Jayaraman, A.; Kumar, P.; Marin, S.; de Atauri, P.; Mateo, F.; M. Thomson, T.; J. Centelles, J.; F. Graham, S.; Cascante, M., Untargeted metabolomics reveals distinct metabolic reprogramming in endothelial cells co-cultured with CSC and non-CSC prostate cancer cell subpopulations. PLOS ONE 2018, 13 (2), e0192175.
40. Krick, T.; Verstraete, N.; Alonso, L. G.; Shub, D. A.; Ferreiro, D. U.; Shub, M.; Sánchez, I. E., Amino Acid metabolism conflicts with protein diversity. Mol Biol Evol 2014, 31 (11), 2905-2912.
41. Chilmonczyk, M. A.; Kottke, P. A.; Stevens, H. Y.; Guldberg, R. E.; Fedorov, A. G., Dynamic Mass Spectrometry Probe (DMSP) for ESI-MS Monitoring of Bioreactors for Therapeutic Cell Manufacturing. Biotechnology and Bioengineering 2018, 0 (ja).
42. Arscott, S., SU-8 as a material for lab-on-a-chip-based mass spectrometry. Lab on a Chip 2014, 14 (19), 3668-3689.
43. Regehr, K. J.; Domenech, M.; Koepsel, J. T.; Carver, K. C.; Ellison-Zelski, S. J.; Murphy, W. L.; Schuler, L. A.; Alarid, E. T.; Beebe, D. J., Biological implications of polydimethylsiloxane-based microfluidic cell culture. Lab on a Chip 2009, 9 (15), 2132-2139.
44. Toepke, M. W.; Beebe, D. J., PDMS absorption of small molecules and consequences in microfluidic applications. Lab Chip 2006, 6 (12), 1484-6.
45. Mukhopadhyay, R., When PDMS isn't the best. Analytical Chemistry 2007, 79 (9), 3248-3253.
46. Kranjc, M.; Miklavčič, D., Electric Field Distribution and Electroporation Threshold. In Handbook of Electroporation, Miklavčič, D., Ed. Springer International Publishing: Cham, 2017; pp 1043-1058.
47. Morshed, B. I.; Shams, M.; Mussivand, T., Electrical Lysis: Dynamics Revisited and Advances in On-chip Operation. Critical Reviews in Biomedical Engineering 2013, 41 (1), 37-50.

### Supplemental Information for Example 1

### Cell Processing Device Fabrication

The cell processing device fabrication sequence can be seen in FIG. 2.7. Process A begins with thermal wet oxidation at 1,100° C. of a 4″ diameter double side polished p-type <100> orientation silicon wafer (1-20 Ω-cm; Polishing Corp of America, Santa Clara, CA) (FIG. 2.7(A1)). This results in a 3 μm thick silicon dioxide layer on both sides of the wafer that is used as an etchant mask for later silicon etching steps. SPR220-7.0 positive photoresist (PR) (Kayaku Advanced Materials, Westborough, MA) is then spin coated as a photolithography layer on the top of the wafer and 360 μm holes are patterned into the SPR220 with darkfield photolithography (FIG. 2.7(A1)); these holes serve as counterbores for direct integration of 360 μm OD capillary inlet/outlet connections. The SPR220 layer is deposited by spinning at 750 rpm with a 1.5 second ramp for 5 seconds immediately followed by 2,500 rpm with a 1.5 second ramp for 40 seconds to deposit an approximately 8 μm thick layer. After spin coating, a 3-minute soft bake on a hotplate at 110° C. is followed by darkfield exposure of the desired pattern at 405 nm wavelength and a dosage of 500 mJ/cm2. The wafer is held for 5 minutes before developing in MicroPosit MF-319 (Kayaku Advanced Materials, Westborough, MA) for approximately 2 minutes. Following a post exposure bake at 100° C. for 15 minutes, the counterbore pattern is etched through the silicon dioxide layer using a CHF3 reactive-ion etch (RIE) (FIG. 2.7(A3)); the remaining SPR220 layer is stripped away with acetone and the wafer is cleaned using an acetone, methanol, isopropanol (AMI) rinse. A new layer of SPR220 is deposited and 100 μm diameter holes are patterned concentric to the counterbore holes. These holes are etched to a depth of approximately 375 μm via a deep reactive-ion etch (DRIE), specifically the Bosch process, to achieve high aspect ratio holes with vertical sidewalls (FIG. 2.7(A5)). The SPR220 is processed using the same procedure as above except the post exposure bake is extended to 3 hours to ensure the PR mask is sufficiently set prior to the longer DRIE.

Following an AMI clean, NR9-1500PY negative photoresist (Futurrex, Franklin, NJ) is then spun on the backside of the wafer (opposite side of counterbore/inlet holes) with 3 second ramp to 3000 rpm for 40 seconds followed by a 3 second ramp down. A soft bake for 1 minute one a hotplate at 150° C. is performed prior to exposure of the electrode design backside aligned with the inlet holes using 375 nm wavelength at an exposure dosage of 775 mJ/cm2. A 1 minute post exposure bake at 100° C. is performed prior to development in RD6 (Futurrex, Franklin, NJ) for approximately 15 seconds followed by a thorough rinse in deionized water (DI); care was taken to not overexposure/overdevelop the electrode design as the negative resist is used as a lift-off layer (FIG. 2.7(A6)). The electrode design is then inset 100 nm into the oxide layer using an RIE etch; this maintains planarity of the wafer surface after deposition of the electrodes to ensure successful bonding (FIG. 2.7(A7)). The electrodes are then deposited via e-beam evaporation (FIG. 2.7(A8)). A 10 nm titanium layer is first deposited as an adhesion layer for the 90 nm gold layer; both layers are deposited at 1 \dot{A}/s. The gold that is deposited on top of the photoresist is then lifted off by soaking the wafer for 10 minutes in an acetone bath followed by a short period of sonication to ensure all gold not in way of the electrodes is fully removed.

MicroPosit SC1827 positive photoresist (Kayaku Advanced Materials, Westborough, MA) is then spun on the backside of the wafer (opposite side of counterbore/inlet holes) with 1.5 second ramp to 1000 rpm for 5 seconds followed by 1.5 second ramp to 3000 rpm for 40 seconds. A soft bake for 1 minute on a hotplate at 115° C. is performed prior to exposure of the channel design aligned with the electrodes using 405 nm wavelength at an exposure dosage of 225 mJ/cm² (FIG. 2.7(A9)). The wafer is held for five minutes following the exposure and then developed in MF-319 for approximately 40 seconds. A hardbake for at least 15 minutes in an oven at 100° C. sufficiently crosslinks the SC1827 to ensure the cell immobilization feature dimensions are preserved as the channel design is etched through the silicon oxide layer via RIE (FIG. 2.7(A10)). SC1827 is used as it forms a thinner layer than SPR220 allowing for more precise patterning of the 3 μm cell immobilization features. The spin parameters above yield an approximately 1.5 μm thick layer of SC1827 that corresponds to a 0.5 aspect ratio for the photolithography step compared to an aspect ratio of 3 if SPR220 were used.

Following the channel oxide etch, the SC1827 is removed by soaking in acetone followed by a 45 second descum. The silicon in way of the channel is then etched to a depth of 27 μm (resulting in a 30 μm deep channel) using a DRIE (the selectivity of the DRIE is high enough that etching of the exposed oxide and gold can be neglected for the 30 μm etch) (FIG. 2.7(A11)). Use of the Bosch process allows for straight sidewalls along the channel and between the cell immobilization features where a 10:1 aspect ratio etch is required. The wafer is then flipped over and the inlet holes are through etched via DRIE (FIG. 2.7(A12)). The counterbore pattern is simultaneously etched during this process as there is no PR mask and the silicon oxide layer was patterned in step A1. This results in a clog resistant inlet/outlet design consisting of a through hole 100 μm in diameter with an outer 360 μm counterbore recessed approximately 250 μm as seen in FIG. 2.5C Process A is completed by sonicating the wafer in acetone to ensure all holes are fully etched through and cleaning the wafer with an AMI rinse.

The bonding process (Process B) begins by scoring the Borofloat wafer in a wafer dicing machine to create two, 80 mm by 24 mm rectangles (FIG. 2.7(B1)). The score is less than 50% of the thickness of the wafer to maintain structural rigidity through the subsequent steps. Following scoring, the Borofloat wafer is soaked in a piranha bath (96% H2SO4 and 30% H2O2 at 3:1 ratio by volume) heated to 125° C. for 1 hour to ensure the surface is free of contaminants. Both the Borofloat and silicon wafer of Process A are then fully dehydrated in an oven at 110° C. to aid in adhesion of the bonding layer. SU8-3005 (MicroChem, Westborough, MA) is then spun on the unscored surface of the Borofloat wafer at 750 rpm with 1.5 second ramp for 5 seconds, 3000 rpm with 1.5 second ramp for 40 seconds, and 4500 rpm with 1 second ramp for 2 seconds. The wafer is then baked on a hotplate for 150 seconds at 90° C. After 150 seconds, the hotplate is turned off and allowed to cool to 65° C. with the wafer without removing the wafer to minimize thermally induced stress in the SU8 layer that could result from the mismatch in thermal properties between SU8 and Borofloat. 1 The SU8 is then flood exposed to 250 mJ/cm² at 365 nm wavelength (FIG. 2.7(B2)); no development or post exposure bake was used. SU8 processing was performed only when the relative humidity was between 30% and 70% to ensure good adhesion of the SU8 layer.2

After exposure, the Borofloat wafer is broken along the scores and the resulting rectangular portions aligned over the process A wafer (SU8 down) to fully cover the channel while allowing the connection pads of the electrodes to remain exposed. After securing the rectangles in place with tape, the wafer stack is bonded in an Obducat NanoImprinter held at 130° C., 10 bar for 30 minutes (Obducat, Burlingame, CA) (FIG. 2.7(B3)). By forgoing the post exposure bake and development, the SU8 layer is not fully crosslinked at the beginning of the bonding process. As pressure and temperature are applied, the SU8 is able to partially reflow between the wafers to seal between surface imperfections while becoming cross-linked by the bonding heat. 1 During bonding, the silicon wafer is placed on the bottom to maximize conductive heating by the heated lower chuck as the thermal conductivity of Si is an order of magnitude higher than Borofloat; this also prevents the full Si wafer from overhanging the rectangular portions of Borofloat which would lead to cracking when pressure was applied. Following dicing to separate the devices, fabrication is completed by inserting inlet and outlet capillaries in the counterbores and securing with UV activated epoxy, Dymax 9-3095-GEL, using a Dymax Light Welder PC-3D system (Dymax, Torrington, CT) (FIG. 2.7(B4)).

### Isolation of Electrical Systems

During preliminary tests, the ESI spray was severely unstable when the lysis circuit was connected. Open inspection, bubbles were observed in the ESI emitter; bubbles are a common issue in ESI workflows as they lead to both intermittent flow and spray and may also electrically emitter if the spray voltage is applied upstream of the emitter rather than to its outer surface. After confirming the fluidic system was sealed and the fluid was sufficiently degassed, and no electrolysis occurred between the electrodes of the cell processing device, it was hypothesized that electrolysis was occurring along the fluidic path, suggesting an electrical sink in the system. Without the electrical lysis circuit connected, it was observed that metal portions of the syringe were in contact with syringe pump in certain orientations. Reorienting the syringe/applying electrical tape solved this issue and allowed the syringe (and system as a whole) to electrically float, eliminating observable electrolysis bubbles and enabling stable ESI spray. With the lysis circuit connected, all upstream components were at 0 potential, suggesting a path(s) to ground through the lysis circuit. The lysis circuit was thus isolated via a double pole single throw switch such that, during normal operation, the ESI voltage is applied with both sides of the lysis circuit being electrically isolated. This prevents the lysis circuit from serving as a path to ground for the ESI voltage during analysis while enabling rapid engagement of the lysis circuit. Upon installation of the switch, the ESI spray displayed excellent stability regardless of where the ESI voltage was applied.

### Fluidic System Design and Characterization

Downstream of the cell processing device, diffusive effects must be considered to prevent excessive dilution of the initial lysate volume. As a first approximation, immediately following lysis, the intracellular biomolecules diffuse into the volume of fluid surrounding the cells, forming a lysate band. As the lysate band flows from the cell processing device to the ESI emitter, it undergoes significant band widening. For low Reynold number, high Peclet number flows commonly seen in active flow microfluidic devices, the extent of band widening is dependent on the effective diffusion coefficient resulting from Taylor-Aris dispersion.3

The equivalent Taylor-Aris dispersion coefficient is calculated according to D_{eff}=Dₒ (1+Pe²/48) where the constant in the denominator is dependent on the geometry and is approximated as 48 for incompressible laminar flow of a Newtonian fluid in a constant cross-section pipe (Hagen-Poiseuille flow). The Peclet number is given according to, Pe=(v*d)/Dₒ where Do is the molecular weight dependent diffusion coefficient (set as 2*10−9 m2/s to represent metabolites), d is the tube inner diameter, and v is the velocity of the fluid for a given flowrate. The effective diffusivity results in growth of the band according to, w=4√ln(2)√(D_{eff}*t). The mean concentration across the resulting bandwidth can then be determined as Cₘ=(Cₒ wₒ)/(wₒ+w) where Co is the initial concentration (here taken as 1 for non-dimensional purposes) and wₒ is the initial bandwidth.

Dispersion effects were investigated for various flowrate, ID, and length combinations relevant the microfluidic workflow. Assuming a 10 cm length of tubing, the Taylor-Aris effective diffusion coefficient rapidly increases (orders of magnitude higher than the absolute diffusion coefficient) for flowrates exceeding 1 μL/hr as shown in FIG. 2.8 (top). Dispersion becomes the controlling method of dilution in this region. The resulting nondimensional dilution factor (Cm/Co) is plotted vs flowrate for varying tube diameters (again assuming a 10 cm length of tubing); the dilution curves collapse for microfluidic tube diameters (10-100 μm ID) at nano-ESI flowrates (1-10 nL/s) (middle). At the experimental flowrate of 30 μL/hr (8.3 nL/s), the internal diameter has minimal impact on the dilution factor and was selected as 50 μm to balance a rapid response against constraints of sustainable pressure drop. Solving the dilution factor vs flowrate for a 100 μm ID tube at varying tube lengths highlights the importance of minimizing tubing length between the cell processing device and the ESI emitter to mitigate dispersion effects. The length of capillary is the controlling parameter with regard to dispersion and must be minimized where possible; minimizing tube length also reduces transit time and pressure drop.

Dispersion modelling also provides useful information as to the delay, duration, and anticipated concentration of analytes in the lysate upon entering the MS. As such, a representative experimental setup was modelled to inform post-processing of MS spectra. Given the low dead volume between the cell immobilization features and device outlet, system modelling began immediately downstream of the device. A 5 cm length of 50 μm capillary leads to a microvalve with 170 nL volume (dispersion in the valve is not considered). A 2.5 cm, 100 μm nominal ID ESI emitter is connected to the valve. The initial bandwidth was determined for a packed condition in which the cells accumulate at the channel restriction (assuming a double layer of 15 μm diameter cells packed 6 abreast in the 100 μm×30 μm channel). The initial dilution upon lysis is given by the intracellular volume divided by the lysate band volume. As the packing density is assumed constant, the initial dilution factor is independent of cell number and equal to approximately 47%. Following dispersion from the lysis location to the emitter, the final dilution factor is approximately 0.4%. Considering a single analyte at an intracellular concentration of 1 μM, the average concentration in the dispersed lysate band will be a mere 4 nM at the emitter tip if extracted from 100 cells. Increasing the number of cells significantly reduces the final dilution (nearly directly proportional), increasing the dilution factor to 4% from 1000 cells. Regardless of cell number, a delay of approximately 55 seconds is expected from the time of lysis to the ESI emitter with the lysate bandwidth corresponding to approximately 20 seconds of spray at a flowrate of 30 μL/hr.

### Application to Intracellular Monitoring

Five of the nineteen amino acids displayed no distinct signal increase at the protonated monoisotopic mass following initial analysis. Additional m/z values were investigated based on fragments reported in open-source ESI-MS databases as well as potential adducts (e.g., Na+, K+, NH4+). Both methionine and arginine displayed distinct signal increases for several of the most common fragments reported in the MassBank Europe database.

Though no common fragments were seen for phenylalanine, the NH4+ adduct displayed a distinct increase compared to the protonated monoisotopic mass; NH4+ is present at high concentration in the buffer.

Individual spectra of representative metabolites are included in FIG. 2.11. Threonine and creatine were both detected at the protonated monoisotopic mass while arginine and spermine were detected as fragments. Tryptophan was not detected at either the protonated monoisotopic or fragment masses. The sensitivity of the system to cell number was characterized by comparing the number of protonated monoisotopic amino acid masses detected. For 200 cells, the concentration of analytes following dispersion is below the limit of detection as no traces display a clear signal at the anticipated lysate elution time point of approximately 3 minutes. For 500 cells, three of the amino acids were detected compared to fourteen of the nineteen for 1500 cells. Comparison of the amino traces for each cell loading can be seen in FIG. 2.12.

### REFERENCES FOR SUPPLEMENTAL INFORMATION FOR EXAMPLE 1

1. Madou, R. M.-D. M. J., SU-8 Photolithography and Its Impact on Microfluidics. In Microfluidics and Nanofluidics Handbook: Fabrication, Implimentation, and Applications, Chakraborty, S. K. M. S., Ed. CRC Press: Boca Raton, FL, 2012; pp 231-268.
2. Birleanu, C.; Pustan, M.; Voicu, R.; Serdean, F.; Merie, V., Humidity influence on the adhesion of SU-8 polymer from MEMS applications. MATEC Web Conf. 2017, 137, 08002.
3. Kirby, B. J., Micro- and Nanoscale Fluid Mechanics: Transport in Microfluidic Devices. Cambridge University Press: Cambridge, 2010.

### Example 2

### Embodiment 1: In-Line Analysis (FIG. 3.1A)

Process flow begins with uptake of cell laden sample directly from culture systems; target number of cells ranges from single cell to 100s of cells with uptake volume given according to target cell count divided by the cell concentration. Upon uptake, the sample plug is infused through the system with cells becoming immobilized in the microfluidic device with extracellular matrix and rinsing buffer passing to waste. Following rinsing, an electrical pulse is applied to lyse the cells and the lysate is directly infused for ESI-MS analysis. The system is reconditioned by back-flowing rinsing buffer via a secondary purge pump to remove cell debris from the microfluidic device.

### Embodiment 2: At-Line Analysis with Periphery Analysis of Rinse (FIG. 3.1A)

The in-line analysis workflow is modified to provide at-line sample preparation in which the sample is gathered off-line and introduced through a fluidic port. Rinsing buffer is directed to periphery analysis systems or directly infused into the mass spectrometer for analysis of the extracellular matrix.

### Embodiment 3: In-Line Lysate Conditioning (FIG. 3.1C)

The workflow is modified to provide additional in-line conditioning of the cell lysate via liquid-liquid, liquid-solid, or capillary electrophoretic separation prior to ESI-MS.

### Design Considerations

The immobilization features for cell capture are designed for a wide range of cell shape, size, and rigidity to enable device operation agnostic to cell type. For analysis of cell like objects (e.g., extracellular vesicles, organelles, bacteria, and viruses), all relevant feature sizes (e.g., spacing in the capture region) are adjusted accordingly to accumulate the given objects. Initial designs consisted of a single step spanning with width of the flow channel as seen in FIG. 3.2A. This design facilitates simple fabrication but is prone to clogging when large numbers of cells are loaded into the device. An alternate design relies on a series of pillars that span the channel height with spacing on the order of single microns as seen FIG. 3.2B. Flow is maintained between the pillars following cell capture, maintaining device operability even for large numbers of loaded cells. The geometry and configuration of the pillars can be modified to maximize capture efficiency while ensuring analyzed cells are able to be purged from the device during reconditioning.

Highly pliable cell types are prone to deform through restrictions much smaller than their nominal diameter and are sensitive to pressure instability in the flow channel as cell capture proceeds. An alternate design was developed that leveraged the variation in cortical tension upon entry of a cell into a restriction. In addition to a series of pillars spanning the channel height, a channel with gap sufficient to remain unclogged at all times is incorporated as seen in FIG. 3.3. Cells that pass through the open channel are captured in the subsequent row by offsetting the position of the always open channel. This design increases cell capture efficiency while preventing pressure instability by eliminating clogs.

The gap size between the pillars is selected such that the pressure drop across each pillar is less than the cortical pressure resulting from a cell entering the restriction. The modified cortical tension was determined according to the Young-Laplace equation

$$Cortical {Tension}_{Restriction} = Cortical {Tension}_{Nominal} ( \frac{1}{{R}_{a}} - \frac{1}{{R}_{b}} )$$

where the small restriction diameter, 2Rₐ, is given by the gap size. As a cell enters the restriction, the resulting bled volume is assumed to be hemispherical. The residual radius, R_{b}, is then determined by approximating the remaining cell volume as a sphere with volume equal to the initial volume minus the bleb volume. The operating regime of such a design can be seen in FIG. 3.3. The pressure drop and cortical tension are plotted against gap size assuming a 10 μm diameter cell with 35 pN/μm nominal cortical tension. The channel was assumed to be 100 μm wide and 25 μm deep with 3 μm square pillars spanning the channel depth. A flow rate of 15 μL/hr was used for the analysis. The region between the two intersecting points of the cortical tension and individual feature pressure drop curves denote the region of successful initial capture (region 1 of FIG. 3.4). In this region, cells that enter the restriction will experience a back pressure insufficient to force them through the restriction.

A second analysis was performed in which a channel sufficiently large to allow cells to pass through while remaining unclogged at all times was designed. The channel configuration should allow for the entire volumetric flow rate to pass-through while still maintaining a pressure drop less than the modified cortical tension of cells captured in the restriction features. The ratio of pressure drop across the channel compared to the pressure drop across individual restriction features should also be minimized such that streamlines are not significantly skewed toward the always open channel prior to the features becoming occupied. This is critical as the device operates at low Reynold number flows in which the inertial component of particle trajectory is negligible. The pressure drop across all restrictions was calculated according to the Darcy-Weisbach equation for fully developed, laminar flow with the entire flow rate used in the pressure drop calculation to simulate all mobilization features having successfully captured cells. Gaps larger than the intersecting value between the individual feature pressure drop and always open channel pressure drop represent configurations in which the diversion of flow is minimal (region 2 of FIG. 3.4). This ensures the individual features will have sufficient flow to direct cells into the restriction rather than exclusively passing through the always open channel. Gaps smaller than the intersecting value between the restriction cortical tension and always open channel pressure drop represent configurations in which the total pressure drop at a given location in the channel will always be less than the pressure threshold for successful capture (region 3 of FIG. 3.4). The intersection of regions 1, 2 and 3 represents the operating regime in which cells will be successfully captured during initial load and remain captured once all sites are occupied.

Following cell loading and capture, the extracellular media must be rinsed from the cells. Experiments were performed to determine a buffer that would not result in uncontrolled osmotic lysis during the rinsing procedure but would also have minimal impact on the MS analysis. Cells in a 12 well plate were stained using a CFSE stain. Media was aspirated and replaced at equal volume with rinsing buffer constituting 1 rinse cycle; cycles were repeated with a target of 3 cycles. Rinsing with native media (control) resulted in negligible reduction in fluorescent signal as seen in Error! Reference source not found.5 1× rinse in water caused near complete release of cytosolic stain within 2 minutes of rinse due to highly hypotonic solution resulting in osmotic lysing; significant fluorescent signal was present in background following osmotic lysis. 3× rinse of water with 0.1% (v/v) acetic acid showed little reduction in signal due to rapid nature of osmotic lysis such that cytosolic contents were released during the first two rinsing cycles. 3× rinse with PlasmaLyte showed negligible reduction in fluorescent signal; PlasmaLyte composition is not amenable to direct infusion ESI-MS. 3× rinse with 150 mM ammonium acetate showed negligible reduction in fluorescent signal although there is possible isolation of fluorescent stain in internal cell features. Given the minimal impact on intracellular state and volatility, ammonium acetate was chosen as the rinsing buffer to ensure cell stability during rinsing while minimizing impact on direct infusion ESI-MS.

Following cell loading and rinsing, a series of electrical pulses is applied across the integrated electrodes to electrically lyse the cells. The lysis circuit seen in FIG. 2.6 employs a switch to electrically isolate the lysis circuit from the ESI circuit/fluidic system when lysis pulses are not needed. This prevents the lysis circuit from serving as a path to ground for the ESI voltage during analysis and prevents uncontrolled electrolysis, the bubbles of which would disrupt ESI spray stability. Leads from the circuit are connected to the exposed electrodes. During the lysis sequence, the leads are held at the same potential until the high voltage transistor (i.e., MOSFET or IGBT) completes the circuit according to the chosen pulse parameters, allowing the voltage to drain across the high resistance (e.g., 10 Mohm) resistor. To enable complete lysis agnostic to cell type, the voltage and pulse duration should be set as high as can be accommodated by the device. This limit is set by the electromigration properties of the electrode and electrochemical breakdown of the flow solution. An alternative electrode configuration in which a thin dielectric layer separates the electrode from the flow channel prevents both electrolysis and Joule heating by inhibiting current flow between the electrodes, allowing for high voltages and long pulse durations to be implemented. For analysis of cell like objects (e.g., extracellular vesicles, organelles, bacteria, and viruses), the relevant lysis procedure (e.g., voltage and pulse parameters) is adjusted accordingly to disrupt the membrane of the given objects.

### Discussion:

Cell processing devices were fabricated as seen in Error! Reference source not found. 2.5A-C. Total dead volume of the device is on the order of nanoliters to reduce dilution effects upon cell lysis. The fabrication sequence is amenable to batch fabrication of robust devices with integrated electrodes and inlet/outlet inface features.

Cell experiments were performed by removing small numbers of cells from a sample suspension, immobilizing the cells within the microfluidic device and performing rinsing cycles to remove all cell culture media. The cells were lysed by a series of electrical pulses that disrupt the cell membrane permanently and instantly. The lysate was then directly infused for nanoESI-MS analysis. Target metabolites representing various metabolite classes including amino acids, vitamins, and fatty acids were selected for peak tracing from existing literature based on conventional HPLC workflows. As seen in FIG. 3.6, the resulting chromatogram traces displayed a distinct increase in a majority of the targeted metabolites with the delay and duration of the lysate signal correlating with dead volume, flowrate, and dispersion calculations. Common fragments also displayed comparable traces, increasing confidence in the metabolites detected. The preliminary targeted analysis implored no additional data processing which would likely elucidate additional analytes via identification of adducts, clusters, and fragments. The results were for direct infusion ESI-MS; additional lysate conditioning prior to MS analysis would only increase the detection and identification capabilities already displayed. These results demonstrate the capability of this analytical workflow to perform rapid, direct-from-culture ESI-MS intracellular analysis of small cell number samples in a robust, reusable microfluidic device.

An at-line embodiment of the workflow was applied for time history monitoring of T-cell activation. Negatively selected CD3+ T-cells were thawed and activated in 6 well plates; unactivated cells were used as the control. Samples were removed from both the activated and unactivated condition over the first 48 hours following thaw. 1 μL sub-samples were introduced into the microfluidic platform for immediate analysis following sample removal. Full scan MS data was collected in positive mode ESI via direct infusion from the cell processing device. The full scan spectra were pre-processed to select features showing significant signal increase in the time period corresponding to elution of the lysate volume. Following alignment of the detected features between samples, dimensionality reduction analyses were performed as seen in FIG. 3.7A-C. PCA of activated and unactivated spectra over the first 48 hours of T-cell culture demonstrate initial grouping followed by distinct separation at later time points. Early time points correspond to cell recovery from thaw with little distinction between the activated and unactivated condition. Between 12 and 18 hours, the activated condition diverges from the unactivated condition along the PC2 axis, remaining distinct at later time points. PCA of the activated condition at early vs late time points shows distinct separation with no overlap between the 95% confidence intervals. PLS-DA of the activated vs unactivated condition at late time points shows distinct separation with no overlap between the 95% confidence intervals. These analyses demonstrate that successful activation can be detected from less than 1000 cells processed within minutes using the sample-to-analysis platform. This is in contrast to conventional intracellular mass spectrometry workflows, secreted protein panels, or surface marker analyses which require 100s of μLs or more per sample and hours or days of sample preparation and analysis time.

Ratios, concentrations, amounts, and other numerical data may be expressed in a range format. It is to be understood that such a range format is used for convenience and brevity, and should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1% to about 5%, but also include individual concentrations (e.g., 1%, 2%, 3%, and 4%) and the sub-ranges (e.g., 0.5%, 1.1%, 2.2%, 3.3%, and 4.4%) within the indicated range. In an embodiment, the term “about” can include traditional rounding according to significant figure of the numerical value. In addition, the phrase “about ‘x’ to ‘y’” includes “about ‘x’ to about ‘y’”.

Unless defined otherwise, all technical and scientific terms used have the same meaning as commonly understood by one of ordinary skill in the art to which this disclosure belongs. Although any methods and materials similar or equivalent to those described can also be used in the practice or testing of the present disclosure, the preferred methods and materials are now described.

Embodiments of the present disclosure will employ, unless otherwise indicated, techniques of separating, testing, and constructing materials, which are within the skill of the art. Such techniques are explained fully in the literature.

It should be emphasized that the above-described embodiments are merely examples of possible implementations. Many variations and modifications may be made to the above-described embodiments without departing from the principles of the present disclosure. All such modifications and variations are intended to be included herein within the scope of this disclosure and protected by the following claims.

1. An analysis system comprising:
a sample introduction system configured to deliver a sample, wherein the sample introduction system includes an inlet and an outlet, wherein the sample includes one or more targeted cargo-containing objects;
an object cargo extraction system, wherein the object cargo extraction system includes an inlet and an outlet, wherein a channel includes one or more capture features positioned between the inlet and the outlet, wherein the one or more capture features are configured to trap the targeted cargo-containing objects in the sample as the sample flows through the channel from the inlet to the outlet, wherein electrodes are positioned adjacent to an area of the channel, wherein the electrodes are configured for electrical lysis of targeted cargo-containing objects present in the area of the channel, wherein the inlet of the object cargo extraction system is in fluidic communication with the outlet of the sample introduction system; and
a detection system, wherein the object cargo extraction system is in fluidic communication with the detection system, wherein the detection system is configured to detect cargo released from the targeted cargo-containing objects.

2. The system of claim 1, wherein the one or more capture features comprises a plurality of pillars, wherein the spacing between the pillars and the pillars and the channel walls is such that the targeted cargo-containing object does not flow past the pillars.

3. The system of claim 1, wherein the plurality of pillars comprises two or more rows of pillars, wherein the spacing between the rows of pillars, the spacing between the pillars in each row, the spacing between the pillars and the channel walls, or a combination of these is such that the targeted cargo-containing object does not flow past the plurality of pillars.

4. The system of claim 1, wherein the plurality of pillars comprises two or more groups of pillars, wherein the spacing between the groups of pillars, the spacing between the pillars in each group, the spacing between the pillars and the channel walls, or a combination of these is such that the targeted cargo-containing object does not flow past the plurality of pillars.

5. The system of claim 1, wherein the one or more capture features comprise one or more permeable membranes, where the targeted cargo-containing objects do not flow past the one or more permeable membranes.

6. The system of claim 1, wherein the one or more capture features and spacing between the features and the walls of the channel have dimensions based on the dimensions of the targeted cargo-containing object, wherein the dimension of the spacing between the capture features and the channel walls relative to the dimensions of the targeted cargo-containing object is based on not allowing the targeted cargo-containing object to flow past the one or more capture features while allowing the cargo released from the targeted cargo-containing objects to flow past the one or more capture features.

7. The system of claim 1, wherein the channel has a length of about 10 μm to 10 cm, a width of about 100 nm to 1 mm, and a height of about 10 nm to 1 mm.

8. The system of claim 1, wherein the sample introduction system is in fluidic communication with a first flow valve, wherein a first setting of the first flow valve is configured to be in communication with the inlet of the object cargo extraction system, wherein the sample in the sample introduction system is flowed through the first valve in the first setting into the object cargo extraction system.

9. The system of claim 1, wherein the sample introduction system is in fluidic communication with a first flow valve, wherein a first setting of the first flow valve is configured to be in communication with the inlet of the object cargo extraction system, wherein a second setting of the first flow valve is configured to be in communication with a structure including the sample, wherein when the first flow valve is in the second setting, the sample introduction system is configured to withdraw the sample from the structure, wherein after withdrawal of the sample, the first flow valve is adjusted to the first setting and the sample is flowed through the first flow valve into the object cargo extraction system.

10. (canceled)

11. The system of claim 1, wherein the detection system is an electrospray ionization mass spectrometry system, wherein the electrospray mass spectrometry system includes an electrospray ionization device, wherein the electrospray ionization device and the electrodes of the object cargo extraction system are electrically decoupled.

12. The system of claim 1, wherein the object cargo extraction system is in fluidic communication with a first waste receiving device in a reverse flow configuration, wherein the object cargo extraction system is in fluidic communication with a purge pump in the reverse flow configuration, wherein a purge fluid is flowed through the object cargo extraction system to the first waste receiving device in the reverse flow configuration.

13. The system of claim 12, wherein the first waste receiving device in fluidic communication with the inlet of the object cargo extraction system when a first flow valve is in a third setting, wherein the first flow valve is positioned between the sample introduction system and the object cargo extraction system,
wherein the purge pump is in fluidic communication with the outlet of the object cargo extraction system when a second flow valve is in a third setting, wherein second flow valve is positioned between the outlet of the object cargo extraction system and the detection system,
wherein the purge pump is in fluidic communication with the first waste receiving device when the first flow valve is in the third setting and the second flow valve is in the third setting such that the purge pump is configured to flow the purge fluid through the object cargo extraction system to the first waste receiving device.

14. The system of claim 1, further comprising a second waste receiving device in fluidic communication with the object cargo extraction system when a second flow valve is in a second setting, wherein the second flow valve is positioned between the object cargo extraction system and the second waste receiving device.

15. The system of claim 1, further comprising a secondary analysis system in fluidic communication with the object cargo extraction system when a second flow valve is in a second setting, wherein the second flow valve is positioned between the object cargo extraction system and the secondary analysis system.

16. The system of claim 1, further comprising a conditioning system positioned between the object cargo extraction system and the detection system, wherein the conditioning system is configured to remove unwanted components present in the fluid sample, retain target components and remove non-target components, introduce signal enhancing components, perform solid phase extraction, perform liquid-liquid extraction, perform electrophoretic separation, perform size exclusion, perform chromatographic separation, perform precipitation based separation, perform electrokinetic separation, perform magnetic separation, perform centrifugation, perform selective component volatilization, perform acoustic separation, perform thermophoresis, or a combination thereof.

17. The system of claim 1, further comprising a conditioning system positioned between the sample introduction system and the object cargo extraction system, wherein the conditioning system is configured to remove unwanted components present in the fluid sample, introduce signal enhancing components, perform solid phase extraction, perform liquid-liquid extraction, perform electrophoretic separation, perform size exclusion, perform chromatographic separation, perform precipitation based separation, perform electrokinetic separation, perform magnetic separation, perform centrifugation, perform selective component volatilization, perform acoustic separation, perform thermophoresis, or a combination thereof.

18. The system of claim 1, further comprising a second object cargo extraction system, wherein the second object cargo extraction system includes an inlet and an outlet, wherein the channel includes one or more capture features positioned between the inlet and the outlet, wherein the one or more capture features are configured to trap the targeted cargo-containing objects in the sample as the sample flows through the channel from the inlet to the outlet, wherein electrodes are positioned adjacent to an area of the channel, wherein the electrodes are configured for electrical lysis of targeted cargo-containing objects present in the area of the channel, wherein the inlet of the object cargo extraction system is in fluidic communication with the outlet of the sample introduction system.

19. The system of claim 18, wherein the object cargo extraction system and the second object cargo extraction system are configured to operate in parallel or in series.

20. (canceled)

21. The system of claim 1, wherein the targeted cargo-containing object are cells, extracellular vesicles, organelles, bacteria, viruses, synthetic membrane-bound particles, synthetic scaffolds to which cells are bound, or a combination thereof.

22. (canceled)

23. A method of analysis, comprising:
introducing a sample to an object cargo extraction system, wherein the sample includes extra-object media and targeted cargo-containing objects, wherein the object cargo extraction system includes a channel, an inlet, an outlet, and a one or more capture features positioned between the inlet and the outlet, wherein the one or more capture features trap the targeted cargo-containing objects in an area of the channel, wherein the object cargo extraction system includes electrodes positioned adjacent the channel, wherein the electrodes are configured for electrical lysis of targeted cargo-containing objects present in the area of the channel;
applying one or more electrical pulse across the electrodes to lyse the targeted cargo-containing objects, wherein the cargo present in the targeted cargo-containing objects pass through the one or more capture features, wherein other debris from lysing the targeted cargo-containing object do not pass through the one or more capture features; and
flowing the cargo out of the outlet to a detection system, wherein the detection system is configured to identify the one or more of the cargo.

24-41. (canceled)
